(19) United States
(12) Patent Application Publication
(10) Pub. No.: US 2026/0256425 A1
(43) Pub. Date: Sep. 3, 2026

(54) MOTION-COMPENSATED LASER SPECKLE CONTRAST IMAGING

(71) Applicant: LIMIS DEVELOPMENT B.V., Leeuwarden (NL)

(72) Inventors: Klaas DIJKSTRA, Leeuwarden (NL); Joost Emiel Marcel CALON, Gorredijk (NL); Arend Jan Wilhelmus VERMEULEN, Drachtster Compagnie (NL); Evert Christiaan Boerma, Leeuwarden (NL)

(73) Assignee: LIMIS DEVELOPMENT B.V., Leeuwarden (NL)

(21) Appl. No.: 18/846,680

(22) PCT Filed: Mar. 3, 2023
(86) PCT No.: PCT/NL2023/050105

§ 371 Date: Sep. 13, 2024

(30) Foreign Application Priority Data
Mar. 17, 2022 (NL) 2031317

(51) Int. Cl. A61B 5/00; A61B 5/026

(52) U.S. Cl. CPC A61B 5/721; A61B 5/0261

(57) ABSTRACT

Methods and systems for motion-corrected and motion-compensated laser speckle contrast imaging are disclosed. The method comprises exposing a target area to coherent first light of a first wavelength, the target area including living tissue, and capturing at least one sequence of images. The at least one sequence of images comprises first speckle images, the first speckle images being captured during the exposure with the first light. The method further comprises determining one or more registration parameters of an image registration algorithm for registering the first speckle images with each other. The method may further comprise, either determining registered first speckle images by registering the first speckle images based on the one or more registration parameters and the image registration algorithm, and determining a combined speckle contrast image based on the registered first speckle images; or determining first speckle contrast images based on the first speckle images, determining registered speckle contrast images by registering the first speckle contrast images based on the one or more registration parameters and the image registration algorithm, and determining a combined speckle contrast image based on the registered first speckle contrast images.

### TECHNICAL FIELD

The disclosure relates to motion-compensated laser speckle contrast imaging, and, in particular, though not exclusively, to methods and systems for motion-compensated laser speckle contrast imaging, a module for motion-compensation in a laser speckle contrast imaging systems and methods and a computer program product enabling a computer system to perform such methods.

### BACKGROUND

Laser speckle contrast imaging (LSCI) provides a fast, full-field, and in vivo imaging method for determining two-dimensional (2D) perfusion maps of living biological tissue. Perfusion can be an indicator for tissue viability and thus may provide valuable information for diagnostics and surgery. For example, during a bowel operation, selection of a high-perfused intervention site may reduce anastomotic leakage.

LSCI is based on the principle that the backscattered light from a tissue illuminated with coherent laser light forms a random interference pattern at the detector due to differences in optical path lengths. The resulting interference pattern is called a speckle pattern, and may be imaged in real-time using a digital camera. Movement of particles inside the tissue causes fluctuations in this speckle pattern resulting in blurring of the speckles in those parts of the images where perfusion takes place.

For example, this blurring may be related to blood flow if the fluctuations are caused by the movement of red blood cells. This way, blood perfusion can be imaged in living tissue in a relatively simple way. Examples of state of the art clinical perfusion imaging schemes by LSCI are described in the review article by W. Heeman et al, ‘Clinical applications of laser speckle contrast imaging: a review’, J. Biomed. Opt. 24:8 (2019). Perfusion by other bodily fluids, e.g. lymph perfusion, may be imaged in a similar way.

LSCI, however, is extremely sensitive to any type of motion. Blurring may not only be caused by movement of blood flow but also any other type of motion such as movement of tissue due to respiration, heartbeat, muscle contraction or to motion of the camera, especially in handheld cameras.

In many medical applications, such as diagnostics and surgery, it is desired that the LSCI system is capable of generating accurate, high-resolution blood flow images, in particular microcirculation images, in real-time, in which motion artefacts are substantially reduced. Hence, during the processing of the raw speckle images, measures are required to minimize motion artefacts so that accurate, high resolution perfusion images can be acquired. This may improve identification of well-perfused and poorly perfused areas, and thus increase diagnosis and treatment outcome.

Various schemes for reducing motion artefacts in speckle images are known in the prior art. For example, WO2020/045015 A1 discloses a laser speckle contrast imaging system which is capable of capturing near-infrared speckle images and white light images of an imaging target. A simple motion detection scheme may include the use of a reference marker on an image target, tracking a feature point in the visible light images, or a change in a speckle shape in a speckle image to determine a global motion vector indicating an amount of movement of an image target between two subsequent images. Speckle contrast images may be generated based on the speckle images and can be corrected for the amount of motion based on the motion vector.

Motion will even be more prominent in handheld LSCI systems, compared to e.g. tripod-supported systems. For example, Lertsakdadet et al, described in their article ‘Correcting for motion artefact in handheld laser speckle images’, Journal of biomedical optics 23 (2), March 2018, a motion compensation scheme for laser speckle imaging using a fiducial marker that is attached to the tissue that needs to be imaged. The use of a marker is not possible in many applications.

Similarly, P. Miao et al, describe in their article ‘High resolution cerebral blood flow imaging by registered laser speckle contrast analysis’, IEEE transactions on bio-medical engineering 57 (5): 1152-1157, a method of producing high-resolution LSCI images by registering raw speckle images based on convolutional filtering and a correlation and interpolation scheme. The registered images are subsequently analysed retrospectively using temporal laser speckle contrast analysis. Such registration of raw speckle images however requires large computational resources and thus are not suitable for accurate real-time imaging applications.

Hence, from the above it follows that there is a need in the art for improved motion-compensated laser speckle contrast imaging schemes. In particular, there is a need in the art for improved methods and systems for laser speckle contrast imaging that allows realization of real-time, robust, markerless, high-resolution perfusion imaging, in particular microcirculation imaging, in which motion artefacts are substantially eliminated, or at least reduced.

### SUMMARY

As will be appreciated by one skilled in the art, aspects of the present disclosure may be embodied as a system, method or computer program product. Accordingly, aspects of the present disclosure may take the form of an entirely hardware embodiment, an entirely software embodiment (including firmware, resident software, micro-code, etc.) or an embodiment combining software and hardware aspects that may all generally be referred to herein as a “circuit,” “module” or “system”. Functions described in this disclosure may be implemented as an algorithm executed by a microprocessor of a computer. Furthermore, aspects of the present disclosure may take the form of a computer program product embodied in one or more computer readable medium(s) having computer readable program code embodied, e.g., stored, thereon.

Any combination of one or more computer readable medium(s) may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium may be transmitted using any appropriate medium, including but not limited to wireless, wireline, optical fiber, cable, RF, etc., or any suitable combination of the foregoing. Computer program code for carrying out operations for aspects of the present disclosure may be written in any combination of one or more programming languages, including a functional or an object oriented programming language such as Java™, Scala, C++, Python or the like and conventional procedural programming languages, such as the “C” programming language or similar programming languages. The program code may execute entirely on the user's computer, partly on the user's computer, as a stand-alone software package, partly on the user's computer and partly on a remote computer, or entirely on the remote computer, server or virtualized server. In the latter scenario, the remote computer may be connected to the user's computer through any type of network, including a local area network (LAN) or a wide area network (WAN), or the connection may be made to an external computer (for example, through the Internet using an Internet Service Provider).

Aspects of the present disclosure are described below with reference to flowchart illustrations and/or block diagrams of methods, apparatus (systems), and computer program products according to embodiments. It will be understood that each block of the flowchart illustrations and/or block diagrams, and combinations of blocks in the flowchart illustrations and/or block diagrams, can be implemented by computer program instructions. These computer program instructions may be provided to a processor, in particular a microprocessor or central processing unit (CPU), or graphics processing unit (GPU), of a general purpose computer, special purpose computer, or other programmable data processing apparatus to produce a machine, such that the instructions, which execute via the processor of the computer, other programmable data processing apparatus, or other devices create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

The flowchart and block diagrams in the figures illustrate the architecture, functionality, and operation of possible implementations of systems, methods and computer program products according to various embodiments. In this regard, each block in the flowchart or block diagrams may represent a module, segment, or portion of code, which comprises one or more executable instructions for implementing the specified logical function(s). It should also be noted that, in some alternative implementations, the functions noted in the blocks may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustrations, and combinations of blocks in the block diagrams and/or flowchart illustrations, can be implemented by special purpose hardware-based systems that perform the specified functions or acts, or combinations of special purpose hardware and computer instructions.

It is an objective of the embodiments in this disclosure to reduce or eliminate at least one of the drawbacks known in the prior art.

In a first aspect, embodiments may relate to a method of motion-compensated laser speckle contrast imaging. The method comprises exposing a target area to coherent first light of a first wavelength, the target area including living tissue, and capturing at least one sequence of images. The at least one sequence of images comprises first speckle images, the first speckle images being captured during the exposure with the first light. The method further comprises determining one or more registration parameters of an image registration algorithm for registering the first speckle images with each other. The method may further comprise, either determining registered first speckle images by registering the first speckle images based on the one or more registration parameters and the image registration algorithm, and determining a combined speckle contrast image based on the registered first speckle images; or determining first speckle contrast images based on the first speckle images, determining registered speckle contrast images by registering the first speckle contrast images based on the one or more registration parameters and the image registration algorithm, and determining a combined speckle contrast image based on the registered first speckle contrast images.

Thus, a sequence of either speckle images or speckle contrast images may be registered or aligned without the use of a marker. The registered images may then be combined into a combined speckle contrast image, having a high resolution and accuracy The combined image may comprise information from a plurality of speckle images, e.g. a pixelwise average, preferably a weighted average, or the combined image can be a single most reliable image in e.g. a moving window of images, for instance the image with the smallest transformation (i.e., closest to the identity transformation using a suitable metric) relative to the subsequent image in a sequence of images.

This way, the images are less sensitive to noise due to motion. Motion can be caused by e.g. motion of the camera, in particular in handheld systems, or motion of the patient, due to e.g. muscle contractions. Thus, the embodiments in this disclosure may enable or improve speckle contrast imaging in a wide range of applications, including, for example, perfusion imaging large bowel tracts, requiring motion of the camera along the entire surface to be imaged, or perfusion imaging of skin burns, where a patient may be unable to remain motionless due to pain.

It is an advantage of the method that the method may be executed (essentially) in real-time using generally available hardware. In some embodiments, there may be a small delay based on, for example, a number of frames (e.g. 20 frames, or 20 frames of sufficient quality), a fixed amount of time (e.g. 1 second), or a time based on physiological properties (e.g. the time for one or two heartbeats or one or two respirations). Such a delay is generally not detrimental to clinical use. Physiological properties may be based on image analysis of the speckle images, the speckle contrast images, and/or the images from the plurality of images, on a predetermined constant based on knowledge on the physiological phenomena or may be based on external input, e.g. from a heart rate monitor.

It is another advantage of the method that the method does not need a fiducial marker to be placed in the field of view. This is especially relevant for areas where placing fiducial markers is undesirable, e.g. when imaging internal organs, burned tissue or brain tissue, or for relatively large image targets that would otherwise require a multitude of fiducial markers or repeatedly replacement of a marker.

As the method corrects and/or compensates for motion of the target area relative to the camera, more reliable perfusion images can be created. In some cases, reliable images may be acquired faster or more easily, for example when a target with irregular motion is imaged, and where an operator would otherwise have to wait for a period with little motion to acquire an image. For example, motion correction may comprise transforming one or more images based on detected apparent motion. Motion compensation may comprise combining a plurality of images, thus increasing the signal to noise ratio.

By registering the first speckle images or the first speckle contrast images, the plurality of first speckle images from the sequence of first speckle images may be used to determine a combined speckle contrast image with an increased contrast and/or spatial resolution, compared to the first speckle contrast images separately. The number of registered speckle images to be combined may depend on the clinical requirements and/or on quality parameters of the first speckle images.

Speckle contrast images may be computed based on the non-registered, and hence untransformed speckle images. Subsequently, the speckle contrast images may be transformed and then combined. The transformation may distort the speckle pattern or parts thereof, for example, speckles may be enlarged, shrunk, or deformed, especially for transformations that are more general than mere translations or rotations. Hence, computing speckle contrast images based on the untransformed speckle images may prevent introducing noise due to the transformation into the speckle contrast images.

Alternatively, the speckle images may be first registered, and hence transformed, and subsequently a speckle contrast image may be computed. This way, a temporal or spatio-temporal speckle contrast may be computed based on two or more registered speckle images. Using temporal or spatio-temporal speckle contrast may lead to a higher spatial resolution. In this embodiment, the images are preferably registered with sub-pixel accuracy.

It is a further advantage of this embodiment that only a single sequence of images is needed: the same sequence of image may be used for (determining registration parameters for) image registration, for determining averaging weights, and for determining a combined laser speckle contrast image or perfusion image. However, in other embodiments, a second sequence of images may be used for determining the registration parameters for image registration, and for determining the averaging weights.

In an embodiment, the determination of the one or more registration parameters is based on a plurality of images, preferably the images in the plurality of images being selected from the first speckle images and/or from images associated with the first speckle images and/or from images derived from the first speckle images or the images associated with the first speckle images.

The images in the plurality of images can be, e.g., images obtained or derived from the respective first speckle images using image processing. For example, the images derived from the first speckle images may represent textures of the first speckle images, normalised versions of the first speckle images, filtered (e.g., blurred or sharpened) first speckle images, or any other suitable image derived from the first speckle images. In other embodiments, described below, separate images are captured and associated with the first speckle images. The images in the plurality of images can also be images derived from the images associated with the first speckle images. As another example, the images in the plurality of images can be images obtained by transforming the first speckle images, image derived from the first speckle images, images associated with the first speckle images, and/or images derived from the images associated with the first speckle images. In an embodiment, the registration parameters are based on a similarity measure of pixel values in one or more pixel groups in each of a plurality of images of the at least one sequence of images, the images in the plurality of images being selected from the first speckle images and/or from images associated with the first speckle images.

The one or more groups of pixels may cover the entire image or only a part thereof. Groups of pixels in different images may have the same or different sizes. For example, where a group of pixels corresponds to a feature, groups corresponding to the same or similar features typically have the same or similar sizes. In a different example, where a group of pixels corresponds to a position in the image, groups of pixels corresponding to the same or a similar position in different images may have different sizes. Matching one or more position-based groups of pixels with, typically, different sizes may also be known as template matching.

An advantage of using groups of pixels is that a group of pixels may typically be matched more reliable than an entire image, in particular when the group is relatively small, compared to the image size. Moreover, if the two images are related by a non-homogeneous transformation, the use of one or more groups of pixels can improve the reliability of the image registration, where a transformation of a group of images may correspond to a local transformation of the image.

In an embodiment, the method further comprises determining transformed images by transforming the first speckle images and/or the images associated with the first speckle images. The transformation may comprise one or more of: a transformation to the frequency domain such as a Fourier transformation, a Mellin transformation, a Laplace transformation, a Radon transformation, and a coordinate transformation such as a log-polar coordinate transformation. In such an embodiment, the registration parameters may be based on a comparison of the transformed images.

In general, the registration parameters may be determined using feature-based, intensity-based and/or frequency-based methods. An advantage of using a feature-based method is that it relates to real-world features, and is therefore less sensitive to image artifacts. Furthermore, features may be selected such that only high-quality data is used to determine the registration parameters, potentially increasing the accuracy and/or robustness. Moreover, feature-based methods can generally cope with both translation and rotations and scaling of an image in a single, in particular when changes are relatively small compared to the image size. In principle, feature-based method may be used with either intensity- or frequency-based methods to determine features. An advantage of intensity-based methods is that they are generally straightforward to implement and computationally cheap to execute. An advantage of frequency-base methods is that information from the entire image is used, making the method less sensitive to local disturbances and image artifacts.

In an embodiment, the transformation is a transformation to a frequency domain, preferably a Fourier transformation. In such an embodiment, the comparison of the transformed images may comprise determining a cross-correlation of the transformed images, determining a transformation of the cross-correlation to the spatial domain, preferably using an inverse Fourier transformation, and determining a peak in the cross-correlation in the spatial domain. A position of determined peak corresponds to a translation between the two images.

In an embodiment, the transformation is a log-polar coordinate transformation; wherein the comparison of the transformed images comprises determining a shift on the transformed images relative to each other; and wherein determining the registration parameters comprises determining a rotation and/or a scaling based on the determined shift. An advantage of using a log-polar coordinate transformation is that a horizontal shift of the transformed image corresponds to a rotation of the untransformed image, and that a vertical shift of the transformed image corresponds to a scaling of the untransformed image. Thus, rotation and scaling can be determined in a relatively straightforward way, in particular global scaling and rotation. A log-polar coordinate transformation is typically applied in combination with a different method to determine a translation of the images.

The combination of a Fourier transformation and a log-polar transformation is sometimes referred to as a Fourier-Mellin transformation.

The determined registration parameters may comprise, e.g., a translation vector, a rotation angle, and scale factor. Based on these registration parameters, the first speckle images may be aligned (transformed) with each other. These registration parameters can also be used to determine weights for a weighted average with which the first speckle images or first speckle contrast images may be combined. In general, it is not necessary to explicitly determine alignment vectors for a multitude of image regions, neither for the image registration itself, nor for weight determination.

An advantage of using large image regions to determine registration parameters, as is typical in, e.g., template matching or frequency-based methods, is that noise may be suppressed, compared to, e.g., methods using a multitude of small regions. This is particularly relevant for registration parameters such as rotation and scaling when determined using log-polar coordinate transformations or Fourier-Melling transformation, as rotation and scaling may be difficult to determine accurately in an untransformed spatial domain.

In an embodiment, the method further comprises determining a plurality of masks for the first speckle images. Each of the plurality of masks is associated with a respective first speckle image. Each of the plurality of masks may associate a reliability score with one or more pixels in the associated first speckle image. In such an embodiment, the determination of the combined speckle contrast may be based, directly or indirectly, on the plurality of masks.

The reliability score may be binary. Alternatively, the reliability score may have more than two potential values, e.g., 255 (byte value), or a floating point value (typically chosen between 0 and 1). Different image types (e.g., speckle images, speckle contrast images, and combined speckle contrast images) may use different mask types, e.g., a binary mask for the speckle images and a byte-valued mask for the speckle contrast images. The mask may be determined for an entire image or only for one or more regions of interest in the image. For example, a speckle contrast value may have a reliability score based on the number of unreliable input pixel values used to determine the speckle contrast. The reliability score can also be based on the registration parameters (for example, based on an amount of motion as determined using, e.g., optical flow). Depending on whether the registration parameters are determined globally (for the entire image), regionally (for image patches or groups of pixels), or locally (for individual pixels), the reliability score can similarly be determined globally, regionally, or locally. Reliability scores from different sources may be combined into a single reliability score.

In principle, the mask may be applied before or after the image registration (and applying the corresponding transformation), and before or after computation of the speckle contrast. Information encoded in the mask may also be used several times during the computation, and may be changed during the computation.

In an embodiment, the method further comprises determining a plurality of registered masks by registering the plurality of masks, based on the one or more registration parameters and the image registration algorithm. In such an embodiment, the determination of the combined speckle contrast may be based on the plurality of registered masks.

In an embodiment, at least one of the plurality of masks is based on an artifact identified in the respective speckle image, preferably a specular reflection artifact. Artifacts may be due to several sources. For example, specular reflection artifacts may occur (especially in endoscopic/laparoscopic set-ups), where pixels or pixel groups are completely saturated, and hence no contrast may be computed. Artifacts can also be due to, e.g., faulty pixel elements in the camera, a stain on the lens collecting the light, et cetera.

The artifacts may be identified by identifying deviating input pixel values, and/or deviating speckle contrast values. For example, pixel values above a predetermined absolute or relative upper threshold value, or values below a predetermined absolute or relative lower threshold value may be marked as deviating pixel values. Additionally or alternatively, pixel contrast values below or above respective absolute or relative lower or upper threshold values may be identified. A relative threshold value may be based on, e.g., an analysis of an environment of the pixel, e.g., a mean or median value or other statistical representation.

In an embodiment, at least one of the plurality of masks is based on pixels identified as not representing living tissue. For example, an image may comprise pixels representing surgical instruments (particularly in an open-surgery set-up), clamps, stitches, gauzes, et cetera. It can be beneficial to not compute a speckle contrast value or perfusion value for these pixels or to at least not display the speckle contrast or perfusion values for these pixels.

In such an embodiment, identifying pixels not representing living tissues may be based on an image recognition algorithm. Such image recognition algorithms are known in the art. The image recognition algorithm may use the speckle images and/or the speckle contrast images as input. In embodiments where the at least one sequence of images also comprises other images than speckle images (e.g., white light images), the pixels not representing living tissues may be identified, additionally or alternatively, in the other images. The identified pixels not representing living tissue may be used to improve perfusion computations in the living tissue and/or to determine overall apparent motion, e.g., by determining an inverse of a perfusion value determined for the pixels not representing living tissue and using that inverse to determine a motion correction value.

In an embodiment, the method further comprises exposing the target area to second light of one or more second wavelengths, preferably coherent light of a second wavelength or light comprising a plurality of second wavelengths of the visible spectrum, wherein the exposure to the second light is alternated with the exposure to the first light or is simultaneous with the exposure to the first light. In this embodiment, the at least one sequence of images comprises a sequence of second images, the second images being captured during exposure with the second light; and the plurality of images is selected from the sequence of second images, each of the second images or a derivative thereof being associated with a first speckle image.

Instead of the raw second images, images derived from the second images may be associated with the first speckle images, e.g., texture images, normalised images, or otherwise pre-processed images.

Thus, in an embodiment, the method of laser speckle contrast imaging comprises alternatingly or simultaneously exposing a target area to coherent first light of a first wavelength and to second light of one or more second wavelengths, preferably coherent light of a second wavelength or light comprising a plurality of second wavelengths of the visible spectrum, the target area preferably including living tissue. The method may further comprise capturing a sequence of first speckle images during the exposure with the first light and a sequence of second images during the exposure with the second light, a speckle image of the sequence of first speckle images being associated with an image of the sequence of second images. One or more registration parameters of an registration algorithm for registering at least a part of the sequence of first speckle images may be determined based on a similarity measure of pixel values of pixel groups in at least a part of the sequence of second images associated with the first speckle images. The method may further comprise determining registered first speckle images by registering the at least part of the sequence of first speckle images based on the one or more registration parameters and the registration algorithm, and determining a combined speckle contrast image based on the registered first speckle images. Alternatively, the method may comprise determining first speckle contrast images based on the at least part of the sequence of first speckle images, determining registered speckle contrast images by registering the first speckle contrast images based on the one or more registration parameters and the registration algorithm, and determining a combined speckle contrast image based on the registered first speckle contrast images.

In the first embodiment defined above, the first light and the second light are the same light with the same wavelength, and the sequence of first speckle images and the sequence of second images are the same sequence of images. However, in an advantageous embodiment, at least one of the one or more second wavelength is different from the first wavelength, and consequently, the second images are different from the first speckle images. A first speckle image and an associated second image can also be different parts of a single image. The images in the sequence of images may be frames in a video stream or in a multi-frame snapshot. In this disclosure, the second images may also be referred to as correction images. Typically, at least one pixel group in each of the at least part of the sequence of second images is used to determine the one or more registration parameters.

Thus, the first wavelength and the capturing of the sequence of first speckle images may be optimised for speckle contrast imaging (e.g. an optimised exposure time) in dependence of the quantity to be measured; while the one or more second wavelength and the capturing of the sequence of second images may be optimised to obtain images that can easily and accurately be registered, e.g. by ensuring a high contrast between anatomical features such as blood vessels and normal tissue. Typical combinations may be speckle images acquired using infrared light and second images acquired using white light, or speckle images acquired using red light and second images acquired using green or blue light, but of course, other combinations are also possible.

In an embodiment, the pixel groups may represent predetermined features in the plurality of images, the predetermined features preferably being associated with objects, preferably anatomical structures, in the target area. Pixel groups may be selected by a feature detection algorithm.

The features may be features associated with physical objects in the target area, e.g. features related to blood vessels, rather than e.g. image features not directly related to objects such as overexposed image parts or speckles. Predetermined features may be determined by e.g. belonging to a class of features, such as corners or regions with large differences in intensity. They may be further determined by e.g. a quality metric, restrictions on mutual distance between features, et cetera. In some embodiments, the neighbourhood of one or more determined features may be used to determine the alignment vectors and/or the transformation.

Determining a displacement based on a relatively small number of features, compared to the total number of pixels, may substantially reduce computation times, while still giving accurate results. This is especially the case for relatively simple motions, where e.g. the entire target area is displaced due to a motion of a camera.

In an embodiment, the method may further comprise filtering the plurality of images with a filter adapted to increase the probability that a pixel group represents a feature corresponding to an anatomical feature. For example, a filter may determine overexposed and/or underexposed areas and/or other image artefacts, and may create a mask based on these areas or artefacts. Thus, determining features related to these areas or artefacts may be prevented.

In an embodiment, determining registration parameters based on a similarity of pixel values of pixel groups may comprise, for each pixel group in an image from the plurality of images, determining a convolution or a cross-correlation with at least part of a different image from the plurality of images. The method may also comprise determining a polynomial expansion to model pixel values in pixel neighbourhoods in the plurality of images, comparing expansion coefficients, and determining alignment vectors based on the comparison.

In an embodiment, determining one or more registration parameters may comprise determining a plurality of associated pixel groups based on the similarity measure, each pixel group belonging to a different image from the plurality of images, determining a plurality of alignment vectors based on positions of the pixel groups relative to the respective images from the plurality of images, the alignment vectors representing motion of the target area relative to the image sensor, and determining the registration parameters based on the plurality of alignment vectors.

By determining alignment vectors for a plurality of features, or pairs of corresponding features, arbitrary movements of the camera relative to the imaging target may be determined and corrected for. The alignment vectors may be used to determine e.g. an affine transformation, a projective transformation, or a homography, which may correct for e.g. translation, rotation, scaling and shearing of an image based on image data alone. Thus, the method does not require information about e.g. distance between camera and target, incident angle, et cetera.

The determination of alignment vectors and/or the determination of the one or more transformations may be based on optical flow parameters, determined using a suitable optical flow algorithm, e.g. a dense optical flow algorithm or a sparse optical flow algorithm.

In an embodiment, determining a combined speckle contrast image may further comprise computing an average of the registered first speckle images, respectively of the registered first speckle contrast images, the average preferably being a weighted average, a weight of an image preferably being based on the registration parameters or based on a relative magnitude of the speckle contrast. Alternatively, combining speckle images or speckle contrast images may comprise filtering the at least part of the first speckle images, respectively first speckle contrast images, with e.g. a median filter or a minimum or maximum filter, et cetera. Weights for weighted averaging may be based e.g. on a quantity derived from the speckle contrast or derived from the alignment vectors.

In an embodiment, computing a combined laser speckle contrast image may comprise computing an average, preferably a weighted average, of the registered sequence of speckle images.

In an embodiment, the average is a weighted average, and masked pixels have a weight equal to zero. If the mask defines a multivalued reliability score, the weight may depend on the reliability score, with pixels having a higher reliability score having a higher weight than pixels having a lower reliability score.

In an embodiment, the method further comprises determining a combined speckle contrast image mask. The combined speckle contrast image mask may indicate whether at most a predetermined percentage of input pixels is masked. For example, the weighted average is only computed if less than a predetermined percentage of input pixels is masked, and wherein pixels are marked as having an invalid pixel value if more than the predetermined percentage of input pixels is masked.

Alternatively, the combined speckle contrast image mask may indicate a reliability score of pixels in the combined speckle contrast image mask. The reliability score may depend on a fraction of masked pixels in the weighted average, and/or on the reliability score of the respective masks.

The speckle contrast or perfusion values are typically shown as an overlay over the captured images. There are various options to treat pixels in the overlay for which no valid perfusion value could be computed. For example, the pixels marked as having an invalid pixel value are removed or rendered as transparent, are assigned an error value, or are assigned a value based on interpolation of surrounding pixel values. Two or more of these options can be combined, e.g., based on the cause of the invalid pixel value and/or based on the size of a region of connected invalid pixel values. For example, invalid pixel regions due to the presence of a non-tissue object (e.g., a surgical instrument) may be shown as transparent, so that the instrument or other non-tissue object can be seen clearly. As a further example, small invalid pixel regions may be filled in using interpolation, to provide a clean image with values that are most likely correct, while large invalid pixel regions may be assigned an error value, indicating that no valid perfusion data was obtained.

If each pixel, or at least each pixel in a relevant area, is assigned reliability score, the reliability score may be rendered by a varying transparency (e.g., using an alpha channel), with reliable pixels having a low transparency (high opacity) and unreliable pixels having a high transparency (low opacity).

In an embodiment, the method may further comprise, for each first speckle image or each first speckle contrast image associated with an image from the plurality of images, determining a transformation size associated with the respective first speckle image or first speckle contrast image based on the plurality of alignment vectors, preferably based on the lengths of the plurality of alignment vectors, and/or on parameters defining the determined transformation. The weighted average may be determined using weights based on the determined transformation size associated with the respective first speckle contrast image, preferably the weight being inversely correlated to the determined transformation size.

A weight based on the size or amount of displacement, or on the size or amount of transformation may be determined quickly for each image, independent of other images. The transformation size may e.g. be based on a norm of a matrix representing the transformation, or the norm of matrix representing a difference between the transformation and the identity transformation. The transformation size may also be based on e.g. a statistically representative measure of the alignment vectors, e.g., the average, median, n-th percentile, or maximum alignment vector length. Images with a large amount of displacement are generally noisier, and may therefore be assigned a lower weight, thus increasing the quality of the combined image.

In an embodiment, the method may further comprise determining, for each first speckle image, a normalised amount of speckle contrast or an amount of change in speckle contrast relative to one or more previous and/or subsequent images in the sequence of first speckle contrast images. The weighted average may be determined using weights based on the determined normalised amount of speckle contrast or the determined change in speckle contrast associated with the respective first speckle contrast image. Alternatively, or additionally, weights may be determined based on a normalised amount of speckle contrast or an amount of change in speckle contrast for the second speckle contrast images.

Weights based on differences or changes in speckle contrast, especially sudden changes, may be indicative for image quality. Typically, speckle contrast, and hence these weights, may be affected by various factors in the entire system, e.g. motion of the camera relative to the target area, movement of fibres or other factors influencing the optical path length or fluctuating lighting conditions. Hence, using weights based on speckle contrast, a higher quality combined image may be obtained. Typically, speckle contrast is determined in arbitrary units, so weights may be determined by analysing a sequence of speckle images. As speckle contrast is inversely correlated with perfusion, speckle contrast-based perfusion units could similarly be used.

In an embodiment, the algorithm may be applied to a predefined region of interest in a field of view of a camera. Such a region of interest may be determined by a user, or may be predetermined. For example, the outer border of the images may be ignored. and/or hidden from view, e.g. to prevent a transformed image border from being visible. Applying the algorithm, or part of the algorithm, to only part of an image may be faster. The region of interest may be transformed based on the determined transformations. In a different embodiment, the algorithm may be applied to the entire image.

In an embodiment, the plurality of images may be the sequence of first speckle images. In such an embodiment, the first wavelength is preferably a wavelength in the green or the blue part of the electromagnetic spectrum. This way, a balance may be struck between a good speckle signal and good visual distinctiveness (i.e., a high contrast of anatomical features, which is to be differentiated from a high speckle contrast), which is advantageous for determining features. Thus, there is no pre-processing step required to increase the contrast of the speckle image.

Alternatively, a first wavelength in the red part of the electromagnetic spectrum may be used, preferably in the range 600-700 nm, more preferably in the range 620-660 nm, or in the infrared part of the electromagnetic spectrum, preferably in the range 700-1200 nm. Depending on the tissue type and imaging parameters such as exposure time, the visual distinctiveness may be sufficient for adequate determination of features. Since red light and infrared light is mostly reflected by red blood cells, these wavelengths result in speckles with a relatively high intensity and are thus very suitable for speckle contrast imaging of blood flow.

As in these embodiments, the first speckle images and the images from the plurality of images are the same images, the images may be acquired with a relatively simple system, requiring only a single light source and a single camera.

In an embodiment, the light of the at least second wavelength may be light of at least a second wavelength different from the first wavelength, preferably coherent light of a predetermined second wavelength, preferably in the green or blue part of the electromagnetic spectrum, preferably in the range 380-590 nm, more preferably in the range 470-570 nm, even more preferably in the range 520-560 nm. Blue or, especially, green light may result in a high contrast or visual distinctiveness, as it is absorbed in the blood vessels much more strongly than by normal tissue. Thus, features, such edges or corners, related to blood vessels may be used to determine the registration parameters,

As the first speckle images themselves are inherently noisy (as far as imaging of anatomical features is concerned), it can be preferable to use second images based on a different wavelength to determine alignment vectors. This way, the first speckle images may be acquired based on light selected to optimise the speckle contrast signal, while the second images may be acquired based on light selected to optimise visual distinctiveness. Such a system is particularly advantageous for imaging tissues where the blood perfusion is relatively deep, e.g. the skin. In such tissues, most of the green or blue light does not penetrate deep enough to interact with the blood cells, resulting in a relatively noise free image.

In an embodiment, the first wavelength is preferably a wavelength in the red part of the electromagnetic spectrum, preferably in the range 600-700 nm, more preferably in the range 620-660 nm, or in the infrared part of the electromagnetic spectrum, preferably in the range 700-1200 nm. Red light and infrared light is mostly reflected by red blood cells, making it very suitable for speckle contrast imaging of blood flow. Infrared light has a larger penetration depth than red light. Red light may be easier to integrate into existing systems, using e.g. a red channel of an RGB camera to acquire a speckle image.

Preferably, the first wavelength is selected to be scattered or reflected by the fluid of interest; for example, red or near-infrared light may be used for imaging blood in blood vessels. Preferably, the first wavelength may be selected based on the required penetration depth. Light with a relatively high penetration depth may allow light scattered by the bodily fluid of interest to be detected with a sufficient signal to noise ratio even at some depth in the imaged tissue.

Preferably, the second wavelength is selected to provide an image with a high visual distinctiveness resulting in consistent features on the tissue surface in the image. For example, green light may be used for imaging blood vessels in internal organs, as green light typically is absorbed much more strongly by blood than by tissues. The light of the second wavelength can be either coherent or incoherent light. The second images may also be based on a multitude of wavelengths, e.g. white light may be used. The light of the second wavelength may be generated by e.g. a second coherent light source. Alternatively, light of the first wavelength and the light of the second wavelength may be generated by a single coherent light source configured to generate coherent light at a plurality of wavelengths.

In an embodiment, the sequence of second images may be a sequence of second speckle images and the method may further comprise determining second speckle contrast images based on the sequence of second speckle images and adjusting or correcting the first speckle contrast images based on changes in speckle contrast magnitude in the sequence of second speckle contrast images.

Multi-spectral coherent correction, also called dual laser correction, may remove or reduce noise in the first speckle contrast images by adjusting the determined speckle contrast in the first speckle images based on a change in determined speckle contrast in sequence of second images. The adjustment may be based on a predetermined correlation between the speckle contrast of the first speckle contrast images and the speckle contrast of the second speckle contrast images.

Multi-spectral coherent correction may advantageously be combined with image registration using second images by using the second images based on the second wavelength both for multi-spectral coherent correction and for image registration. In such an embodiment, the second wavelength preferably has a relatively small penetration depth. This way the second image may comprise information that mostly relates to the surface of the target area. This is especially true for tissues with little perfusion close to the surface, such as the skin, scar tissue, and some tumour types.

In an embodiment, the method may further comprise dividing each image in the at least part of the sequence of first speckle images, respectively first speckle contrast images, and each image in the plurality of images into a plurality of regions, preferably disjoint regions. Preferably, the regions in the image from the plurality of images correspond to the regions in the associated first speckle image, respectively first speckle contrast image. Determining registration parameters may comprise determining registration parameters for each region, and determining a sequence of registered first speckle images, respectively first speckle contrast images, may comprise registering each region of the first speckle image, respectively first speckle contrast image, based on the transformation based on the corresponding region in the image from the plurality of images.

The regions may be determined based on e.g. the geometry of the image, e.g. a grid of rectangular or triangular regions, or based on image properties, e.g. light intensity or pixel groups that appear to belong to an anatomical structure.

This way, local movements in part of the image may be corrected, leading to a higher quality combined image. Local movements are typically caused by motion in the target, e.g. due to the person moving, respiration, heartbeat, or muscle contraction such as peristaltic motion in the lower abdomen. The regions may be as small as single pixels. If a weighted average is used to combine two or more images, weights may be assigned to each region separately and/or to the image as a whole. Combining images may likewise be region based, or be done on an image-by-image basis.

In an embodiment, the target area may comprise a perfused organ, preferably perfused by a bodily fluid, more preferably perfused by blood and/or lymph fluid, and/or may comprise one or more blood vessels and/or lymphatic vessels. The method may further comprise computing a perfusion intensity, preferably a blood perfusion intensity or a lymph perfusion intensity, based on the combined speckle image.

The method may further include post-processing the images, e.g. thresholding, false colouring, overlying on other images, e.g. white light images, and/or displaying the combined image or a derivative thereof.

In a second aspect, embodiments may be related to a hardware module for an imaging device, preferably for a medical imaging device. The hardware module may comprise a first light source a first light source for exposing a target area to coherent first light of a first wavelength, the target area preferably including living tissue. The hardware module may further comprise an image sensor system with one or more image sensors for capturing at least one sequence of images, the at least one sequence of images comprising first speckle images, the first speckle images being captured during the exposure with the first light. The hardware module may further comprise a computer readable storage medium having computer readable program code embodied therewith, and a processor, preferably a microprocessor, more preferably a graphics processing unit, coupled to the computer readable storage medium, wherein responsive to executing the computer readable program code, the processor is configured to: determine one or more registration parameters of an image registration algorithm for registering the first speckle images with each other, the registration parameters being based on a similarity measure of pixel values of pixel groups in a plurality of images, the images in the plurality of images being selected from the first laser speckle images or being associated with the first speckle images, the registration parameters preferably defining one of: a homography, a projective transformation, or an affine transformation; and determine registered first speckle images by registering the first speckle images based on the one or more registration parameters and the image registration algorithm, and determine a combined speckle contrast image based on the registered first speckle images; or determine first speckle contrast images based on the first speckle images, determine registered speckle contrast images by registering the first speckle contrast images based on the one or more registration parameters and the image registration algorithm, and determine a combined speckle contrast image based on the registered first speckle contrast images.

In an embodiment, the hardware module may comprise a second light source for illuminating, simultaneous or alternatingly with the first light source, the target area with light of at least a second wavelength, different from the first wavelength. The at least one image sensor may be configured to capture a sequence of second images, the second images being captured during exposure with the second light. The plurality of images may be selected from the sequence of second images, each of the second images being associated with a first speckle image.

The image sensor system may comprise a first image sensor for capturing the sequence of first images, and a second image sensor for capturing the sequence of second images, or a single image sensor for capturing both the sequence of first images and the sequence of second images.

In an embodiment, the hardware module may further comprise a display for displaying the combined speckle image and/or a derivative thereof, preferably a perfusion intensity image. Alternatively or additionally, the hardware module may comprise a video output for outputting the combined speckle image and/or the derivative thereof.

The image sensor system may comprise a first image sensor for capturing images of the first wavelength and a second image sensor for capturing images of the at least second wavelength. The first image sensor and the second image sensor may be the same image sensor, different parts of a single image sensor, e.g. red and green channels from an RGB camera, or different image sensors. The module may further comprise optics to guide light from the first light source and from the optional second light source to a target area and/or to guide light from the target area to the first and second image sensors.

The disclosure is further related to a medical imaging device, preferably a endoscope, a laparoscope, a surgical robot, a handheld laser speckle contrast imaging device or an open surgical laser speckle contrast imaging system comprising such a hardware module.

In a further aspect, the disclosure is related to a computation module for a laser speckle imaging system, comprising a computer readable storage medium having at least part of a program embodied therewith, and a processor, preferably a microprocessor, more preferably a graphics processing unit, coupled to the computer readable storage medium, wherein responsive to executing the computer readable storage code, the processor is configured to perform executable operations. The executable operations may comprise:
receiving at least one sequence of images, the at least one sequence of images comprising first speckle images, the first speckle images having been captured during exposure of a target area to coherent first light of a first wavelength, the target area including living tissue; determining one or more registration parameters of an image registration algorithm for registering the first speckle images with each other, the registration parameters being based on a similarity measure of pixel values of pixel groups in a plurality of images of the at least one sequence of images, the images in the plurality of images being selected from the first speckle images or being associated with the first speckle images, the registration parameters preferably defining one of: a homography, a projective transformation, or an affine transformation; and determining registered first speckle images by registering the first speckle images based on the one or more registration parameters and the image registration algorithm, and determining a combined speckle contrast image based on the registered first speckle images; or determining first speckle contrast images based on the first speckle images, determining registered speckle contrast images by registering the first speckle contrast images based on the one or more registration parameters and the image registration algorithm, and determining a combined speckle contrast image based on the registered first speckle contrast images.

Such a computation module may e.g. be added to an existing or new medical imaging device such as a laparoscope or an endoscope, in order to improve laser speckle contrast imaging, in particular perfusion imaging. In an embodiment, the method steps described in this disclosure may be executed by a processor in a device for coupling coherent light into an endoscopic system. Such a device may be coupled between a light source and a video processor of an endoscopic system, and an endoscope, e.g. a laparoscope, of the endoscopic system. The coupling device may thus add laser speckle imaging capabilities to an endoscopic system. Such a coupling device has been described in more detail in Dutch patent application NL 2026240, which is hereby incorporated by reference.

In an embodiment, the at least one sequence of images comprises a sequence of second images, the second images being captured during exposure with second light, the second light having one or more second wavelengths, preferably the second light being coherent light of a second wavelength or the second light comprising a plurality of second wavelengths of the visible spectrum, wherein the exposure to the second light is alternated with the exposure to the first light or is simultaneous with the exposure to the first light. The plurality of images may be selected from the sequence of second images, each of the second images being associated with a first speckle image.

The disclosure may also relate to a computer program or suite of computer programs comprising at least one software code portion or a computer program product storing at least one software code portion, the software code portion, when run on a computer system, being configured for executing any of the method steps described above.

The disclosure may further relate to a non-transitory computer-readable storage medium storing at least one software code portion, the software code portion, when executed or processed by a computer, is configured to perform any of the method steps as described above.

The embodiments will be further illustrated with reference to the attached schematic drawings. It will be understood that the disclosure is not in any way restricted to these specific embodiments.

### BRIEF DESCRIPTION OF THE DRAWINGS

The following description of the figures of specific embodiments is merely exemplary in nature and is not intended to limit the present teachings, their application or uses.

FIG. 1A schematically depicts a system for motion-compensated laser speckle contrast imaging according to an embodiment and FIG. 1B-E depict flow diagrams for laser speckle contrast imaging according to embodiments;

FIG. 2A-C depict a raw speckle image, a laser speckle contrast image based on the raw speckle image, and a perfusion image based on the laser speckle contrast image;

FIG. 3A-D depict flow diagrams for motion-compensated laser speckle contrast imaging according to embodiments;

FIGS. 4A and 4B depict flow diagrams for motion-compensated laser speckle contrast imaging according to embodiments;

FIG. 5A-F depict methods for determining registration parameters according to embodiments;

FIG. 6A-D depict methods for determining registration parameters according to embodiments;

FIGS. 7A and 7B depict flow diagrams for motion-compensated laser speckle contrast imaging combining more than two raw speckle images according to an embodiment;

FIG. 8 depicts a flow diagram for computing a corrected laser speckle contrast image according to an embodiment;

FIGS. 9A and 9B schematically depict determining motion-compensated speckle contrast images based on a weighted average, according to an embodiment; and

FIG. 10 is a block diagram illustrating an exemplary data processing system that may be used for executing methods and software products described in this application.

### DETAILED DESCRIPTION

Laser speckle contrast images may be based on spatial contrast, temporal contrast, or a combination. In general, using spatial contrast leads to a high temporal resolution but a relatively low spatial resolution. Additionally, individual images may suffer from e.g. quality loss due to motion or lighting artefacts, resulting in an image quality that may vary from image to image. On the other hand, using a temporal contrast is associated with a relatively high spatial resolution and a relatively low temporal resolution. However, the quality of temporal contrast may be strongly affected by motion of the target relative to the camera, which may lead to pixels being incorrectly combined. Mixed methods may share some advantages and disadvantages of both methods.

In this disclosure, speckle images may also be referred to as raw speckle images to better differentiate between (raw) speckle images and speckle contrast images. The term ‘raw speckle image’ may thus refer to an image representing a speckle pattern, with pixels having pixels values representing a light intensity. Raw speckle images may be unprocessed images or (pre-) processed images. The term ‘speckle contrast image’ may be used to refer to a processed speckle image with pixels having pixel values representing a speckle contrast magnitude, typically a relative standard deviation over a predefined neighbourhood of the pixel.

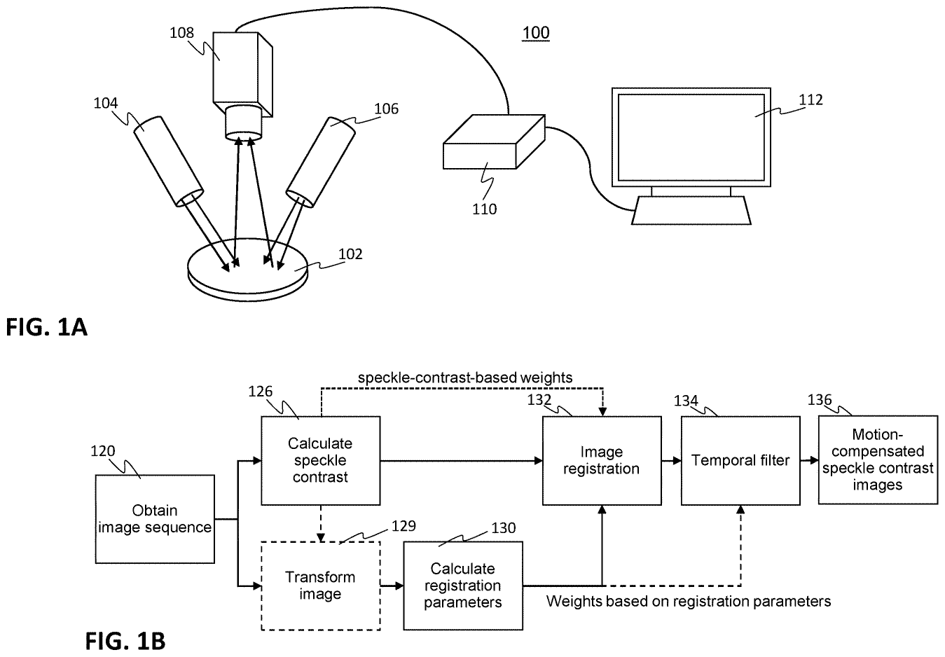

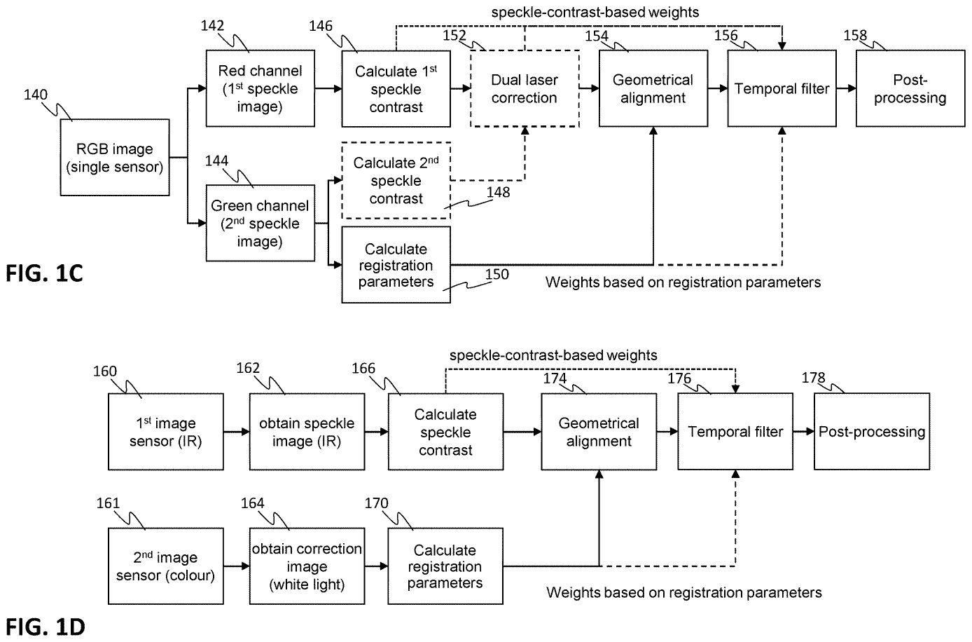

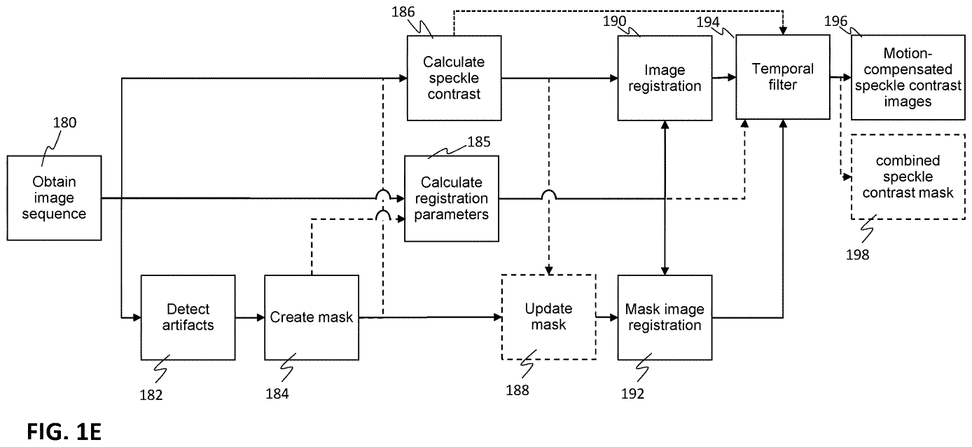

FIG. 1A schematically depicts a system 100 for motion-compensated laser speckle contrast imaging according to an embodiment. The system may comprise a first light source 104 for generating coherent light, e.g. laser light, of a first wavelength for illuminating a target area 102. The target is preferably living tissue, e.g. skin, bowel, or brain tissue. The first wavelength may be selected to interact with a bodily fluid which may move through the target, for instance blood or lymph fluid. The first wavelength may be in the red or (near) infrared part of the electromagnetic spectrum, e.g. in the range 600-700 nm, preferably in the range 620-660 nm, or in the range 700-1200 nm. The first wavelength may be selected based on the bodily fluid of interest and/or the tissue being imaged. The first wavelength may also be selected based on the properties of an imaging sensor. Depending on, inter alia, exposure time, size of the imaged area, speckle size, and image resolution, different quantities of interest may be imaged, e.g. flow in individual large or small blood vessels, or microvascular perfusion of a target area.

In an embodiment, the system may further comprise a second light source 106 for generating light of at least a second wavelength, preferably comprising light of the green part of the electromagnetic spectrum, for illuminating the target area 102. The at least second wavelength may be selected to comprise a wavelength that creates images with a high visual distinctiveness, that is, a high contrast of anatomical features. The second wavelength may be selected based on the tissue in the target area. In general, the light of the at least second wavelength may be coherent light or incoherent light, and may be monochromatic, e.g. blue or green narrow-band imaging light, or polychromatic light, e.g. white light. In other embodiments, only the first light source is used. In the embodiment depicted in FIG. 1C, the light of the at least second wavelength is monochromatic coherent light. The second wavelength may be generated by e.g. a second coherent light source. Alternatively, light of the first wavelength and the light of the second wavelength may be generated by a single coherent light source configured to generate coherent light at a plurality of wavelengths.

The system may further comprise one or more image sensors 108 for capturing images associated with light of the first wavelength and, when applicable, images associated with light of the at least second wavelength, the light of the first and at least second wavelengths having interacted with the target in the target area. In a different embodiment, the system may comprise a plurality of cameras, for example a first camera for acquiring first raw speckle images associated with the first wavelength and a second camera for acquiring correction images associated with the second wavelength. The system may furthermore comprise additional optics, e.g. optical fibres, lenses, or beam splitters, to guide light from the one or more light sources to the target area and from the target area to the one or more image sensors.

When the target is illuminated with coherent light, a laser speckle pattern may be formed through self-interference. The images may be received and processed by a processing unit 110. Examples will be described in further detail with reference to FIG. 1B-D. The processing unit may output processed images in essentially real-time to e.g. a display or to a computer 112. The processing unit may be a separate unit or may be part of the computer. The processed images may be displayed by the display or computer.

In an embodiment, an endoscope or laparoscope may be used to guide light to the target area and to acquire images. The one or more light sources, the one or more image sensors, the processing unit and the display may e.g. be part of an endoscopic system.

FIG. 1B-E depict flow diagrams for motion-compensated laser speckle contrast imaging according to an embodiments. Alternatives that are mentioned with respect to one of these embodiments can also be applied to the other embodiments, unless where such combination would result in a contradictory description.

FIG. 1B depicts a flow diagram for motion-compensated laser speckle contrast imaging according to an embodiment. In this embodiment, only coherent light of the first wavelength generated by the first light source 104 is used, which can be e.g. green or, preferably, red light. The one or more image sensors 108 is a single image sensor, typically a monochromatic image sensor optimised or at least suitable for the used wavelength. As was indicated above and will be shown in the examples of FIGS. 1C and 1D below, other embodiments may use different configurations.

In a first step 120, a sequence of raw speckle images is obtained, e.g. captured or received from an external source. In this example, these speckle images are also used as correction images. Based on each first raw speckle image, a first speckle contrast image may be computed 126. Speckle contrast may be determined in any suitable way, e.g. by a convolution with a predetermined convolution kernel, or by determining the relative standard deviation of pixel value intensities in a sliding window. As the speckle contrast is correlated with perfusion, perfusion unit weights may be determined based on the speckle contrast values of the first speckle contrast images.

In a single-wavelength embodiment, either the raw speckle images or the speckle contrast images may be used as correction images. In an optional step 129, the correction images may be transformed. Based on the optionally transformed correction images, registration parameters may be calculated. For example, in each correction image, positions of predetermined object features may be determined. Based on the positions of the predetermined object features in two or more correction images, alignment vectors identifying motion of the target area may be determined, for example using an optical flow algorithm. A (sparse) optical flow algorithm may be used to determine alignment vectors based on selected features. Other embodiments may use e.g. a dense optical flow algorithm; in that case, there is no need to determine features.

Based on the alignment vectors, transformations for registering images in the second sequence of images may be determined. Preferably, the transformations are selected from the class of homographies, from the class of projective transformations, or from the class of affine transformations. Optionally, optical flow weights may be determined based on the alignment vectors or on parameters defining the transformation.

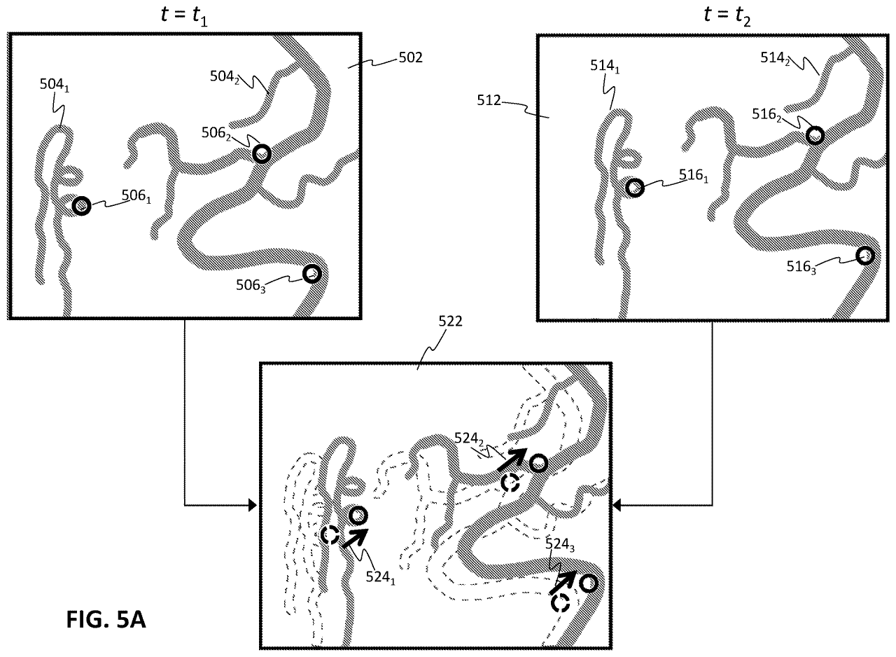

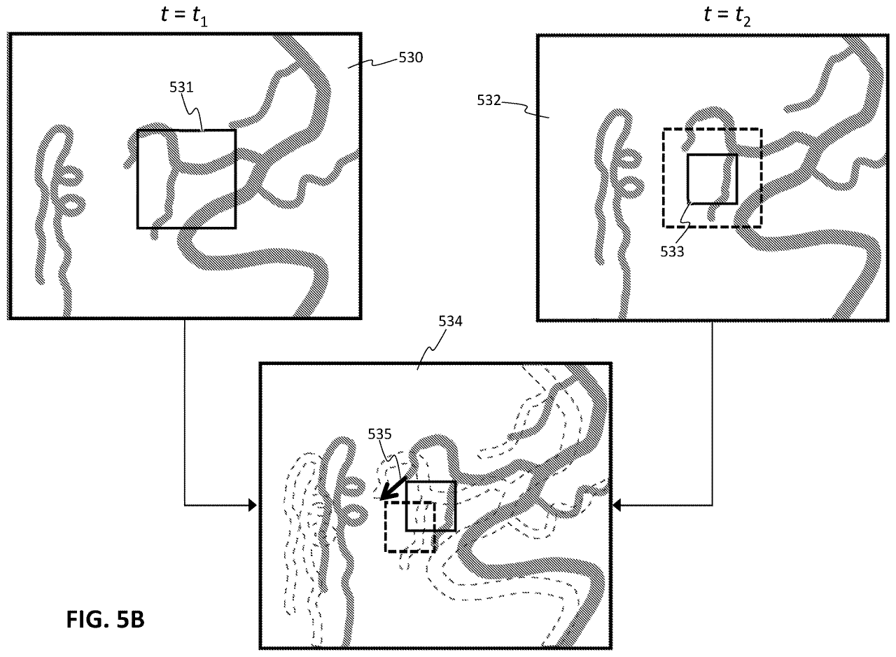

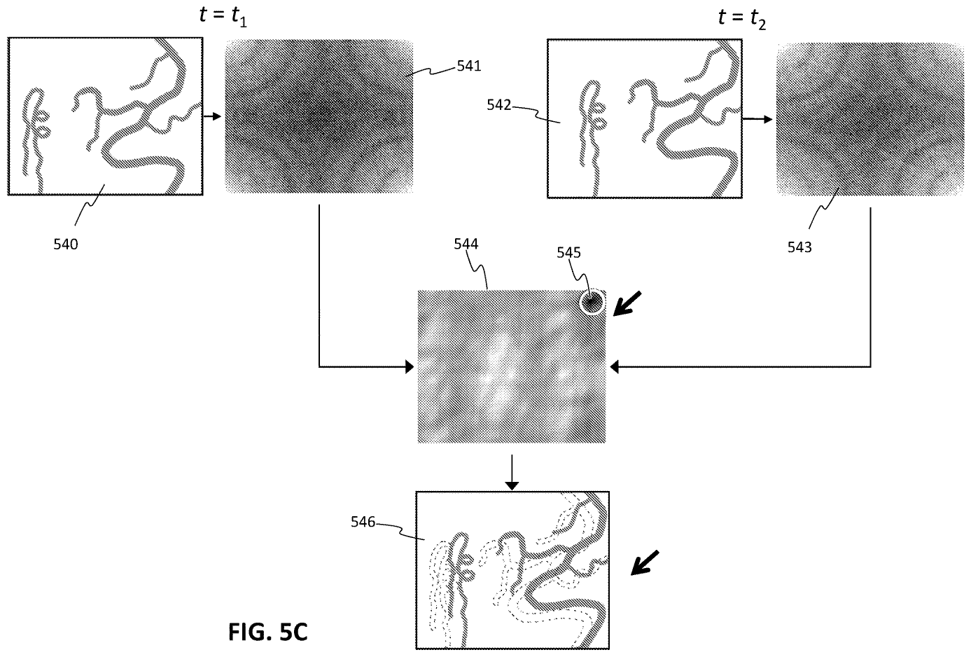

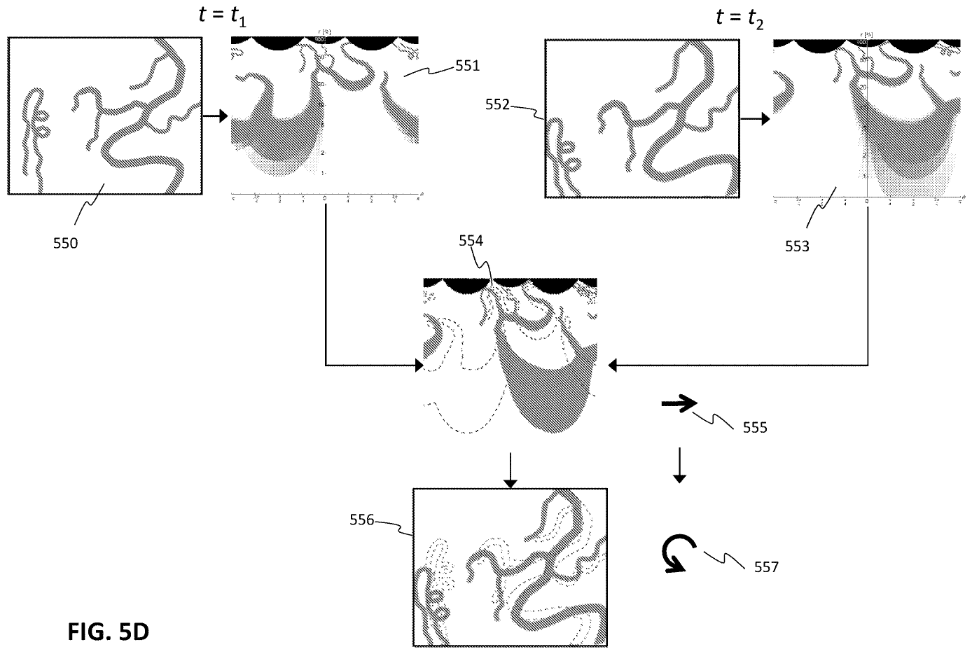

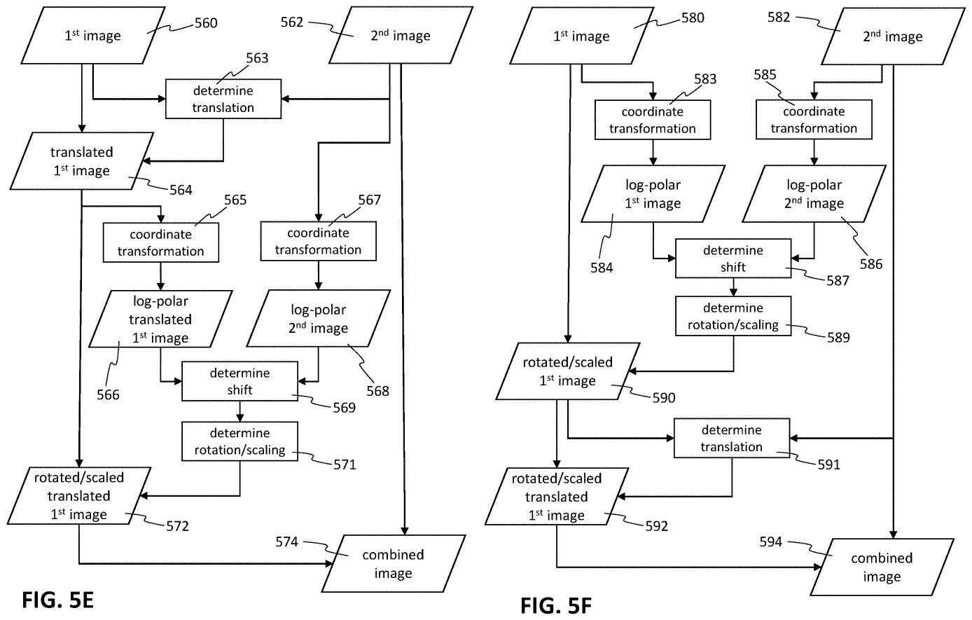

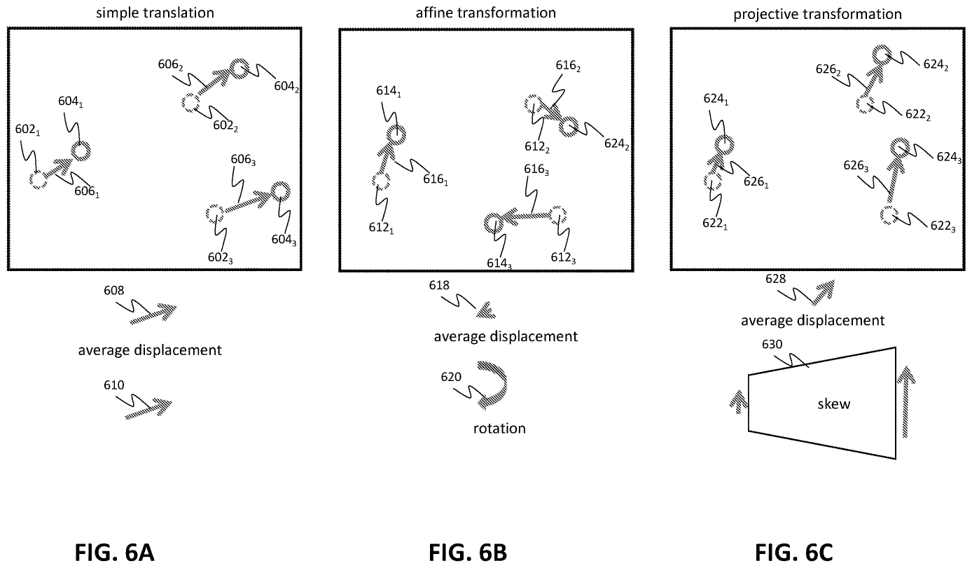

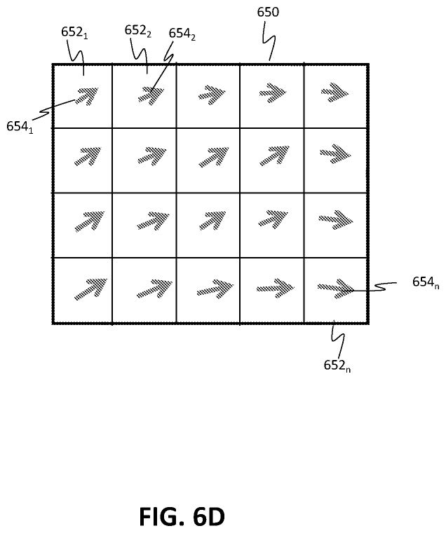

Alternatively, the registration parameters can be computed in any other suitable way, for instance using template matching, phase matching, matching in a log-polar coordinate system, et cetera. Various examples of suitable methods to determine registration parameters will be described in more detail below with reference to FIGS. 5A-D and FIG. 6.

The determined registration parameters may then be used to register 132, or geometrically align, the speckle contrast images with each other, resulting in registered first speckle contrast images. In an alternative embodiment, the raw speckle images may be registered before computing the speckle contrast. However, as the image registration may affect the pixel values and hence the contrast, such an embodiment is less preferred.

Subsequently, the registered first speckle contrast images may be combined 134, e.g., using a temporal filter. The temporal filter may comprise averaging a plurality of first speckle contrast images. The averaging may be weighted averaging, with the weights being based on the optical flow weights and/or based on perfusion unit weights. In an alternative embodiment, the registered raw speckle images may be combined using the temporal filter, and a speckle contrast image may be determined based on the combine draw speckle image. This results in motion-compensated speckle contrast images 136.

Afterwards, the combined first raw speckle images may be post-processed, e.g. converted to perfusion values, thresholded to indicate areas with high or low perfusion, overlain on a white light image of the target area, et cetera. The post-processing may be done by e.g. the processing unit 110 or the computer 112. FIG. 1C depicts a flow diagram for motion-compensated laser speckle contrast imaging according to an embodiment. In the depicted embodiment, the light of the first wavelength generated by the first light source 104 is red light, and the light of the at least second wavelength generated by the second light source 106 is coherent green light. The one or more image sensors 108 are a single sensor comprising red, green, and blue channels. The first wavelength and the second wavelength are selected to minimise crosstalk. The signal in the red channel caused by the green light is substantially smaller than the signal caused by the red light; and the signal in the green channel caused by the red light is substantially smaller than the signal caused by the green light. As was indicated above, other embodiments may use different configurations.

In a first step 140, a sequence of RGB images is received. A first sequence of first raw speckle images may be extracted 142 from the red channel of sequence of the RGB image, and a second sequence of correction images may be extracted 144 from the green channel of the RGB image. The sequence of correction images may be a second sequence of second raw speckle images. Each first raw speckle image may be associated with the correction image extracted from the same RGB image. In other embodiments, the first raw speckle images and the correction images may be acquired by different cameras, by different sensors of a multi-sensor camera (e.g., a 3CCD camera), by other (colour) channels of a single camera (e.g., a YUV camera), or, if the target is illuminated alternately with light of the first wavelength and light of the at least second wavelength, the images may be acquired alternately by a single monochrome camera.

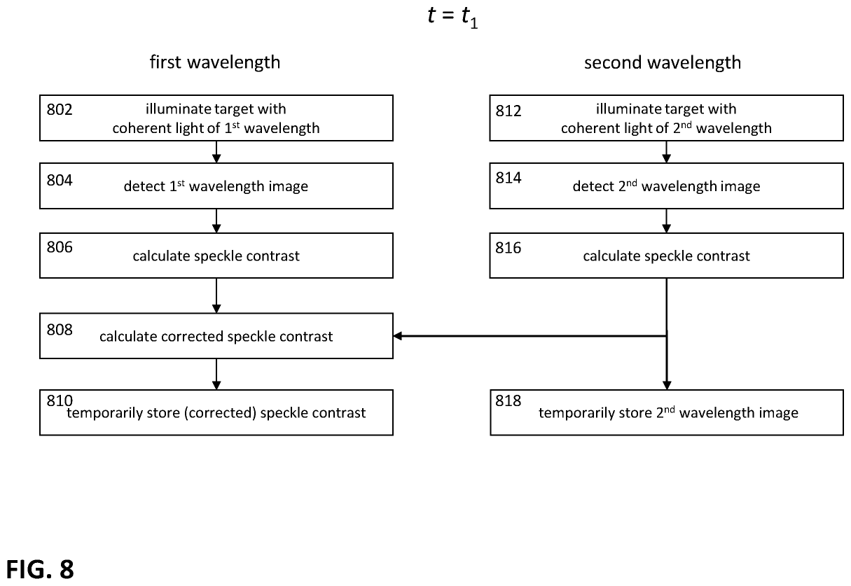

Based on each first raw speckle image, a first speckle contrast image may be computed 146. Optionally, based on each second raw speckle image, a second speckle contrast image may be computed 148. Speckle contrast may be determined in any suitable way, e.g. by a convolution with a predetermined convolution kernel, or by determining the relative standard deviation of pixel value intensities in a sliding window. As the speckle contrast is correlated with perfusion, perfusion unit weights may be determined based on the speckle contrast values of the first speckle contrast images. The second speckle contrast image may optionally be used to correct 152 the first speckle contrast image, as will be described in more detail with reference to FIG. 8. In that case, the corrected speckle contrast values may be used to determine perfusion unit weights.

Based on the correction images (i.e., in this embodiment, the second raw speckle images), registration parameters may be computed 150. For example, positions of predetermined object features may be determined. Based on the positions of the predetermined object features in two or more correction images, alignment vectors identifying motion of the target area may be determined, for example using an optical flow algorithm. A (sparse) optical flow algorithm may be used to determine alignment vectors based on selected features. Other embodiments may use e.g. a dense optical flow algorithm; in that case, there is no need to determine features. Alternatively, the registration parameters can be computed in any other suitable way, for instance using template matching, phase matching, matching in a log-polar coordinate system, et cetera. Some of these examples may comprise determining a transformed image based on the correction image. Various examples of suitable methods to determine registration parameters will be described in more detail below with reference to FIGS. 5A-D and FIG. 6.

Based on the registration parameters alignment vectors, transformations for registering images in the second sequence of images may be determined. Preferably, the transformations are selected from the class of homographies, from the class of projective transformations, or from the class of affine transformations. Optionally, optical flow weights may be determined based on the alignment vectors or on parameters defining the transformation.

The determined registration parameters may then be used to register 154, or geometrically align, the first speckle contrast images associated with the correction images. Subsequently, the registered first speckle contrast images may be combined 156, e.g. using a temporal filter. The temporal filter may comprise averaging a plurality of first speckle contrast images. The averaging may be weighted averaging, with the weights being based on the optical flow weights and/or based on perfusion unit weights.

In a final step 158, the combined first raw speckle images may be post-processed, e.g. converted to perfusion values, thresholded to indicate areas with high or low perfusion, overlain on a white light image of the target area, et cetera. The post-processing may be done by e.g. the processing unit 110 or the computer 112.

FIG. 1D depicts a flow diagram for motion-compensated laser speckle contrast imaging according to an embodiment. In the depicted embodiment, the light of the first wavelength generated by the first light source 104 is infrared light, but other colours such as red, green, or blue are also possible. The light of the at least second wavelength generated by the second light source 106 is (incoherent) white light. An advantage of using infrared light and white light is that both may be used simultaneously without substantially affecting each other

As depicted, the one or more image sensors 108 are two image sensors in two cameras, an infrared camera and a colour camera. This can be practical if the laser speckle imaging is added to a system already comprising a colour camera, for instance in an open surgery setting. Additionally, having dedicated image sensors may allow separate optimisation of hardware and/or equipment parameters such as exposure time. As was indicated above, other embodiments may use different configurations. For example, in some embodiments, a single camera may be used to capture both the infrared and the (white light) colour images. An advantage of using a single camera is that the infrared images and the colour images are automatically aligned and associated with each other.

In a first step 160, a first sequence of first images is captured by the infrared camera, which may be stored as a sequence of speckle images 162. This first sequence may be stored by the image processor as a sequence of raw speckle images. In a second step 161, a second sequence of second images is captured by the colour camera. This second sequence may be stored 164 as a sequence of correction images. Each raw speckle image may be associated with one or more correction images. Preferably, each raw speckle image is associated at least with the correction image that was captured closest in time to, preferably simultaneous with the raw speckle image. Preferably, the frame rates of the first and second cameras are chosen such as to allow a straightforward association, e.g. by selecting one frame rate as a integer multiple of the other frame rate.

Based on each raw speckle image in the sequence of speckle images, a speckle contrast image may be computed 166. Speckle contrast may be determined in any suitable way, e.g. by a convolution with a predetermined convolution kernel, or by determining the relative standard deviation of pixel value intensities in a sliding window. As the speckle contrast is correlated with perfusion, perfusion unit weights may be determined based on the speckle contrast values of the first speckle contrast images.

Based on the correction images (i.e., in this embodiment, the white light images), registration parameters may be computed 170. For example, positions of predetermined object features may be determined. Based on the positions of the predetermined object features in two or more correction images, alignment vectors identifying motion of the target area may be determined, for example using an optical flow algorithm. A (sparse) optical flow algorithm may be used to determine alignment vectors based on selected features. Other embodiments may use e.g. a dense optical flow algorithm; in that case, there is no need to determine features. Alternatively, the registration parameters can be computed in any other suitable way, for instance using template matching, phase matching, matching in a log-polar coordinate system, et cetera. Some of these examples may comprise determining a transformed image based on the correction image. Various examples of suitable methods to determine registration parameters will be described in more detail below with reference to FIGS. 5A-D and FIG. 6.

Based on the alignment vectors, transformations for registering images in the sequence of correction images may be determined. Preferably, the transformations are selected from the class of homographies, from the class of projective transformations, or from the class of affine transformations. Optionally, optical flow weights may be determined based on the alignment vectors or on parameters defining the transformation.

The determined registration parameters may then be used to register 174, or geometrically align, the first speckle contrast images associated with the correction images. Since, in this embodiment, two cameras are used, the two cameras do not share a field of view and frame of reference. Consequently, registering the speckle contrast images based on the registration parameters determined using the correction images may comprise applying a transformation to the registration parameters to account for this change in frame of reference. If the cameras are positioned in a fixed position relative to each other, this transformation may be predetermined. Otherwise, the transformation can be determined based on e.g. image processing of calibration images or markers. Markers can be either natural or artificial.

Subsequently, the registered first speckle contrast images may be combined 176, e.g. using a temporal filter. The temporal filter may comprise averaging a plurality of first speckle contrast images. The averaging may be weighted averaging, with the weights being based on the optical flow weights and/or based on perfusion unit weights.

In a final step 178, the combined first raw speckle images may be post-processed, e.g. converted to perfusion values, thresholded to indicate areas with high or low perfusion, overlain on a white light image of the target area, et cetera. The post-processing may be done by e.g. the processing unit 110 or the computer 112.

FIG. 1E depicts a flow diagram for motion-compensated laser speckle contrast imaging according to an embodiment. A step 180 comprises obtaining an image sequence, e.g., as described above with reference to FIG. 1B. Furthermore, in a step 182, artifacts are detected in the obtained images. In this context ‘artifacts’ should be understood broadly, and may refer to any pixel value that may adversely affect tissue perfusion computations. Artifacts may be due to several sources. For example, specular reflection artifacts may occur (especially in endoscopic/laparoscopic set-ups), where pixels or pixel groups are completely saturated, and hence no contrast may be computed. Artifacts can also be due to, e.g., faulty pixel elements in the camera, a stain on the lens collecting the light, et cetera.

The artifacts may be identified by identifying deviating input pixel values, and/or deviating speckle contrast values. For example, pixel values above a predetermined absolute or relative upper threshold value, or values below a predetermined absolute or relative lower threshold value may be marked as deviating pixel values. For example, fully saturated pixels (i.e., pixels having a maximal pixel value) may be excluded, but it may be beneficial to also exclude pixels that are almost saturated, e.g., having a pixel value equal to or higher than 99% of the maximum pixel value.

A relative threshold value may be based on, e.g., an analysis of an environment of the pixel, e.g., a mean or median value or other statistical representation. For example, pixels that deviate more than a specified amount from a regional median, or that deviate more than a predetermined number of (regional) standard deviations from a regional mean, may be identified as artifacts. Alternatively, a (Gaussian) blur may be applied to the image in order to determine a background value relative to which the relative threshold may be applied. The region for determining outliers is typically chosen substantially larger than the window for computing spatial speckle contrast.

Furthermore, a boundary region around these pixels may be included in the mask, for example by growing the regions with identified pixels. The size of the border region may be based on the size of the region over which the spatial contrast is computed. For example, if the border region is at least equal in size to the radius of the window for spatial contrast, spatial contrast may be computed without taking the mask into account and all speckle contrast values that are based on identified pixels will be masked.

Additionally or alternatively, pixel contrast values below or above respective absolute or relative lower or upper threshold values may be identified. Certain artifacts may lead to a very low or very high speckle contrast value. Furthermore, the size and/or shape of a region with very high or low computed speckle contrast value may be used to identify artifacts.

In this context, an image artifact may also refer to pixels not representing living tissue. For example, an image may comprise pixels representing surgical instruments (particularly in an open-surgery set-up), clamps, stitches, gauzes, et cetera. As these objects are not living tissue, they are (generally) not perfused. Therefore, the speckle contrast images may be used to identify these kinds of artifacts, in addition to or instead of the speckle images. In embodiments where the at least one sequence of images also comprises other images than speckle images (e.g., white light images), the pixels not representing living tissues may be identified, additionally or alternatively, in the other images.

Image recognition algorithms may also be used to identify artifacts. This is particularly useful for artifacts having a known shape or other known or learnable visual properties.

In some embodiments, the detection of artifacts is limited to one or more regions of interest in the image. In that case, also the subsequent steps may be limited to such a region of interest.

In a step 184, a mask is created for each image in the sequence of input images, based on the detected artifacts for that image. The mask associate a reliability score with one or more pixels in the corresponding input image. This is typically a binary mask, indicating which pixels are deemed unreliable. However, a multivalued (i.e., non-binary) may also be used; for example, a pixel identified as unreliable may be surrounded by a region of increasingly reliable pixels.

In some cases, a plurality of masks may be determined for each input image, e.g., one mask representing deviating pixel values, and one mask representing non-tissue objects. Using multiple masks allows for different downstream treatment of masked pixels. Alternatively, a multivalued mask may be used, with different values indicating different sources of uncertainty or error.

In a step 185, the registration parameters are calculated as described above. It may be beneficial to compute the image registration parameters based on the masked image, to prevent fitting on image artifacts. For example, some feature-based image registration algorithms may try to register overexposed spots or specular reflection artifacts; using a mask excluding these artifacts may force the registration algorithm to register on living-tissue features instead.

In a step 186, the speckle contrast is computed. In some cases, the speckle contrast computation takes the computed mask into account. Depending on the implementation, when the input for a speckle contrast computation comprises one or more masked pixels, these masked pixels may be ignored, or the computation may be skipped or an error value assigned. The treatment of masked pixels may depend on the absolute or relative number of masked pixels in the input.

In some cases, further unreliable pixels may be determined based on the computed speckle contrast, e.g., based on deviating speckle contrast values.

Thus, in an optional step 188, the mask is updated (or a new mask is created) based on the computed speckle contrast.

In principle, it is possible to apply the mask directly to the speckle image or to the speckle contrast image, i.e., replacing pixel values in the speckle (contrast) image with mask values. However, it is often beneficial to keep the mask in a separate image (or separate image layer). However, in that case, care must be taken that the mask matches the image. Therefore, in a step 192, the mask is transformed using the same transformation algorithm and parameters as are used for the image registration of the speckle contrast image in step 190.

In a step 194, the temporal filter is applied to the speckle contrast images as described above. For example, a pixel-by-pixel weighted average may be computed, in which the weight of each pixel may depend on, e.g., the image registration parameters and/or the local or global speckle contrast in the image. Additionally (or even alternatively), the weight may depend on the mask. In particular, masked pixels may have a weight equal to zero. If the mask defines a multivalued reliability score, the weight may depend on the reliability score, with pixels having a higher reliability score having a higher weight than pixels having a lower reliability score. Thus, a sequence of motion-compensated speckle contrast images may be determined 196, based on the sequence of speckle contrast images and associated masks.

In an optional step 198, a combined speckle contrast image mask is determined. The combined speckle contrast image mask may indicate whether at most a predetermined percentage of input pixels is masked. For example, the weighted average is only computed if less than a predetermined percentage of input pixels is masked, and wherein pixels are marked as having an invalid pixel value if more than the predetermined percentage of input pixels is masked.

Alternatively, the combined speckle contrast image mask may indicate a reliability score of pixels in the combined speckle contrast image mask. The reliability score may depend on a fraction of masked pixels in the weighted average, and/or on the reliability score of the respective masks. The mask may also be based on other weight factors that enter the temporal filter, such as local or global registration parameters and/or local or global speckle contrast values.

The speckle contrast or perfusion values are typically shown as an overlay over the captured images. There are various options to treat pixels in the overlay for which no valid perfusion value could be computed. For example, the pixels marked as having an invalid pixel value are removed or rendered as transparent, are assigned an error value, or are assigned a value based on interpolation of surrounding pixel values. Two or more of these options can be combined, e.g., based on the cause of the invalid pixel value and/or based on the size of a region of connected invalid pixel values. For example, invalid pixel regions due to the presence of a non-tissue object (e.g., a surgical instrument) may be shown as transparent, so that the instrument or other non-tissue object can be seen clearly. As a further example, small invalid pixel regions may be filled in using interpolation, to provide a clean image with values that are most likely correct, while large invalid pixel regions may be assigned an error value, indicating that no valid perfusion data was obtained.

If each pixel, or at least each pixel in a relevant area, is assigned reliability score, the reliability score may be rendered by a varying transparency (e.g., using an alpha channel), with reliable pixels having a low transparency (high opacity) and unreliable pixels having a high transparency (low opacity).

In an embodiment, the method steps described in this disclosure may be executed by a processor in a device for coupling coherent light into an endoscopic system. Such a device may be coupled between a light source and a video processor of an endoscopic system, and an endoscope, e.g. a laparoscope, of the endoscopic system. The coupling device may thus add laser speckle imaging capabilities to an endoscopic system. Such a coupling device has been described in more detail in Dutch patent application NL 2026240, which is hereby incorporated by reference.

In an alternative embodiment, the method steps described in this disclosure may be applied in an open surgical setting, possibly in combination with a pre-existing imaging system.

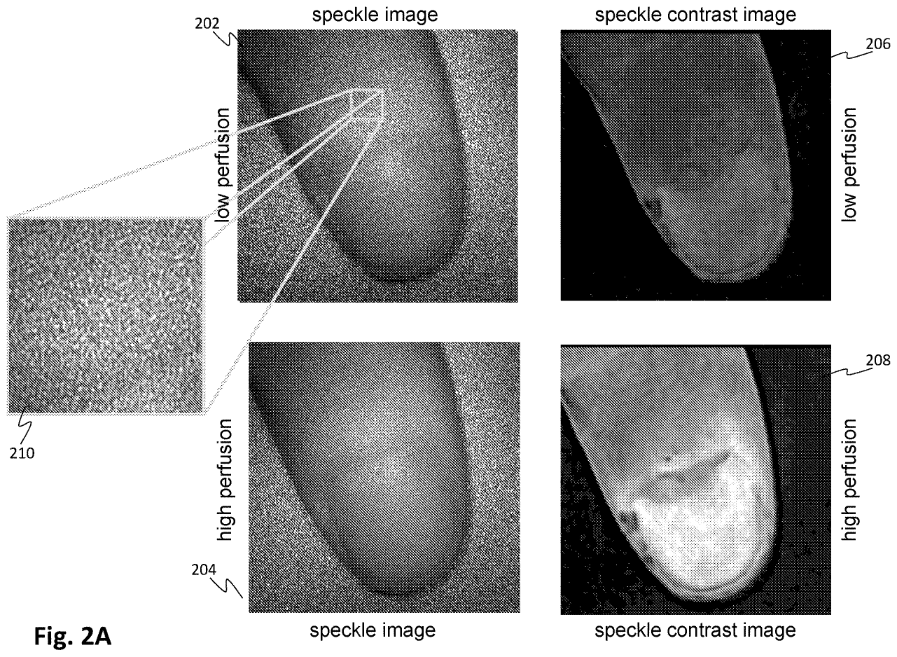

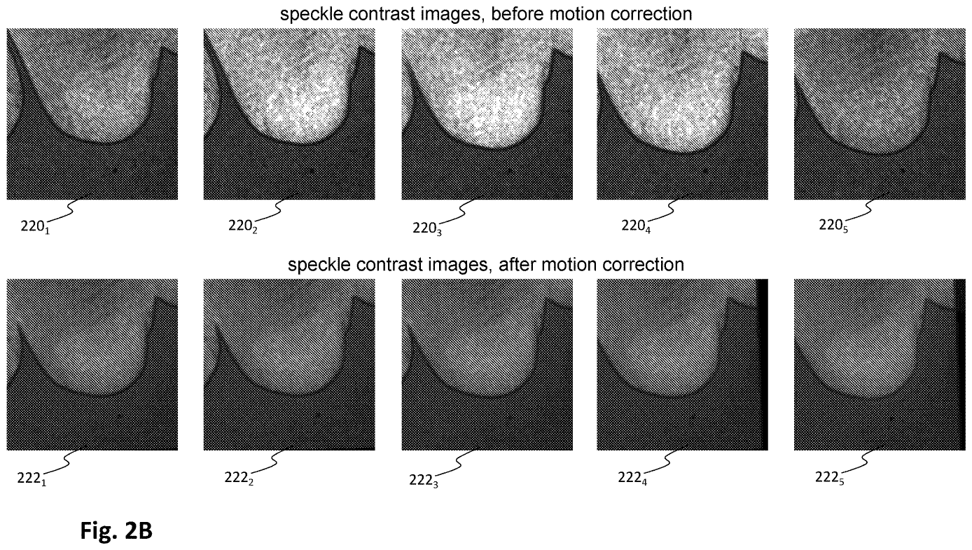

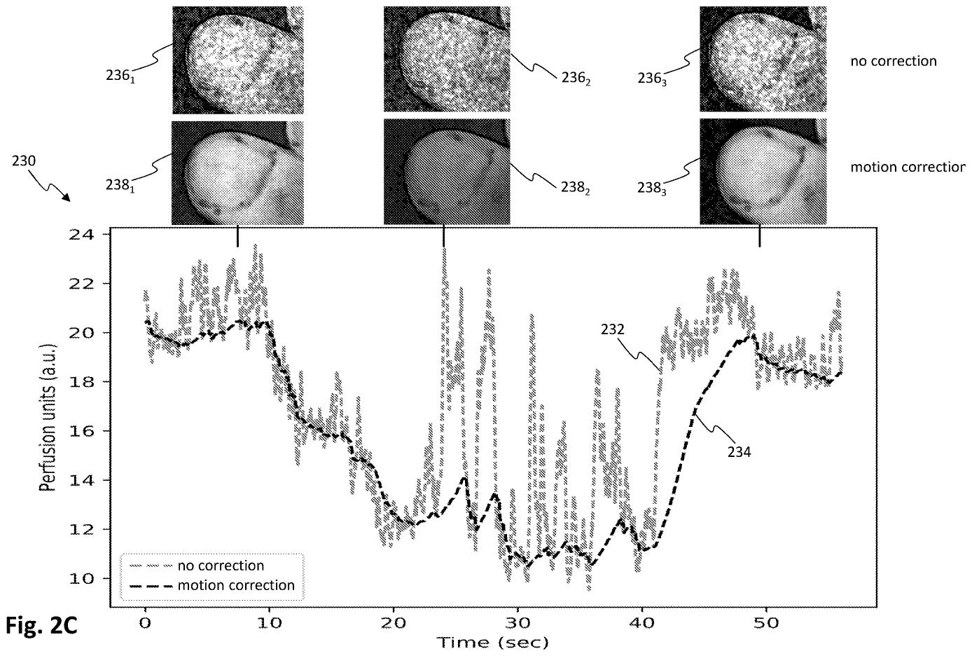

FIG. 2A depicts a raw speckle images, and laser speckle contrast images based on the raw speckle images, of a target with low perfusion and a target with high perfusion. Images 202 and 204 are raw speckle images of the tip of a human finger, including a nail bed. When image 202 was obtained, blood flow through the finger was restricted, resulting in a low blood perfusion of the finger (artificial ischemia). When image 204 was obtained, blood flow was unrestricted, resulting in a much higher blood perfusion, compared to the previous situation. For a human viewer, it is difficult to see differences in the speckle pattern associated with the difference in perfusion. A zoomed-in part 210 of image 204 is also shown, displaying the speckle structure in more detail.

Images 206 and 208 are speckle contrast images based on images 202 and 204, respectively. A light colour represents a low contrast, and hence a high perfusion, while a dark colour represents a high contrast, and hence a low perfusion. In these images, the difference in perfusion is immediately clear, especially in the nail bed where the blood flow occurs relatively close to the surface.

FIG. 2B depicts a series of laser speckle contrast images of a low perfusion target exhibiting motion, before motion correction and after motion correction according to an embodiment. Images 220₁₋₅ are speckle contrast images of the tip of a human finger, including a nail bed. During the acquisition of images 220₂₋₄, the finger moved, leading to loss of contrast due to finger motion. If a user is interested in blood flow, the low speckle contrast in images 220₂₋₄, represented by a light colour, may be considered a motion artefact. Images 222₁₋₅ are based on the same raw speckle images as images 220₁₋₅, respectively, but have been corrected by a motion correction algorithm according to an embodiment.

FIG. 2C depicts speckle contrast images from a series of speckle contrast images and a graph representing a perfusion level based on the series of speckle contrast images. Graph 230 depicts a perfusion measurement of a nailbed, showing a first curve 232 representing the perfusion determined based on uncorrected measurements, and a second curve 234 representing the perfusion determined based on measurements corrected with a motion correction algorithm as described in this disclosure. Around the time mark of 10 seconds, blood flow to the finger is artificially restricted, and about 30 seconds later, the restriction is removed. In particular between the time range of 23 till 38 seconds, the uncorrected perfusion measurement show a number of motion artefacts, where the perfusion seems to sharply rise and fall again.

The figure further shows three exemplary speckle contrast images before (images 236₁₋₃) and after (images 238₁₋₃) processing with a motion correction and compensation algorithm based on a single wavelength. In these images, a light colour represents a low speckle contrast, and hence a high perfusion, while a dark colour represents a high speckle contrast, and hence a low perfusion. The three uncorrected images appear more or less the same, making it difficult for a user to recognise a time (or in other applications, a region) with low or high perfusion. By contrast, the motion corrected images display a clear difference between the (middle) image acquired during restriction of the blood flow and the other images with unrestricted blood flow, allowing a user to select a time or place with a high perfusion.

Additionally, anatomical structures such as the edges of the finger and the nailbed can more easily be recognised in the motion-compensated image, while details may be hard to recognise in the uncorrected images due to their grainy nature. This further facilitates interpretation of the images by a user.

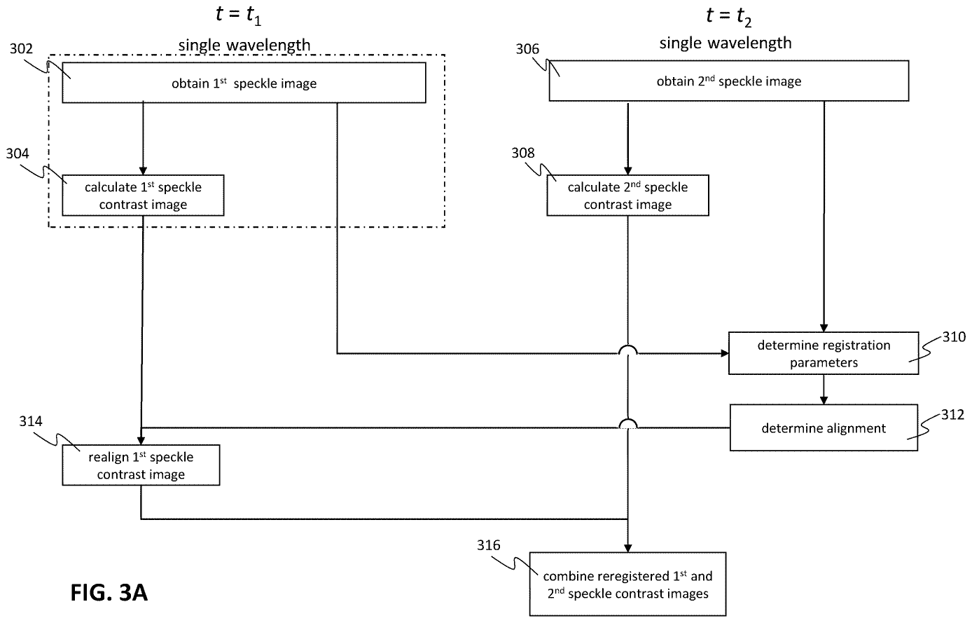

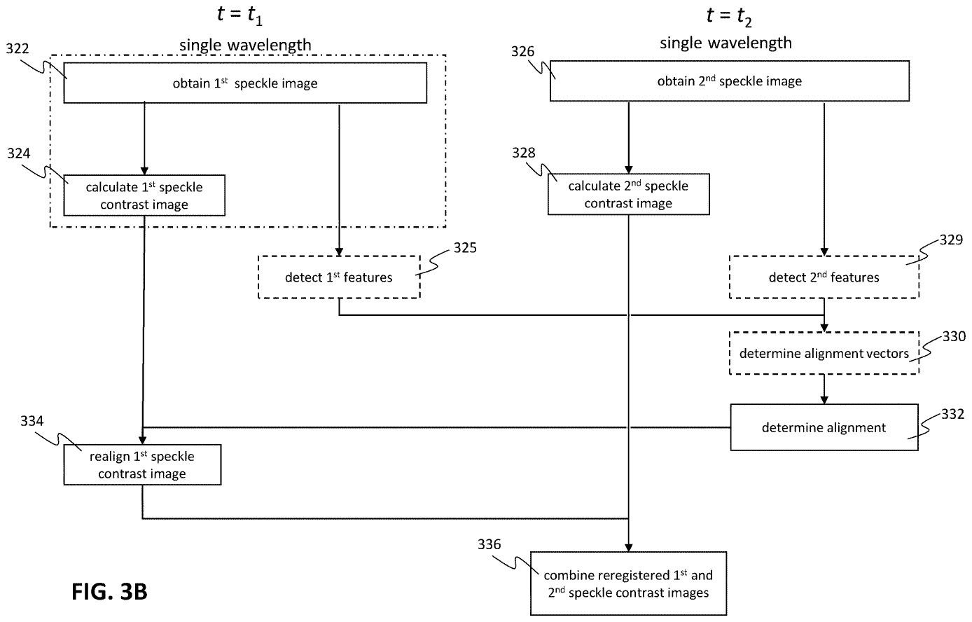

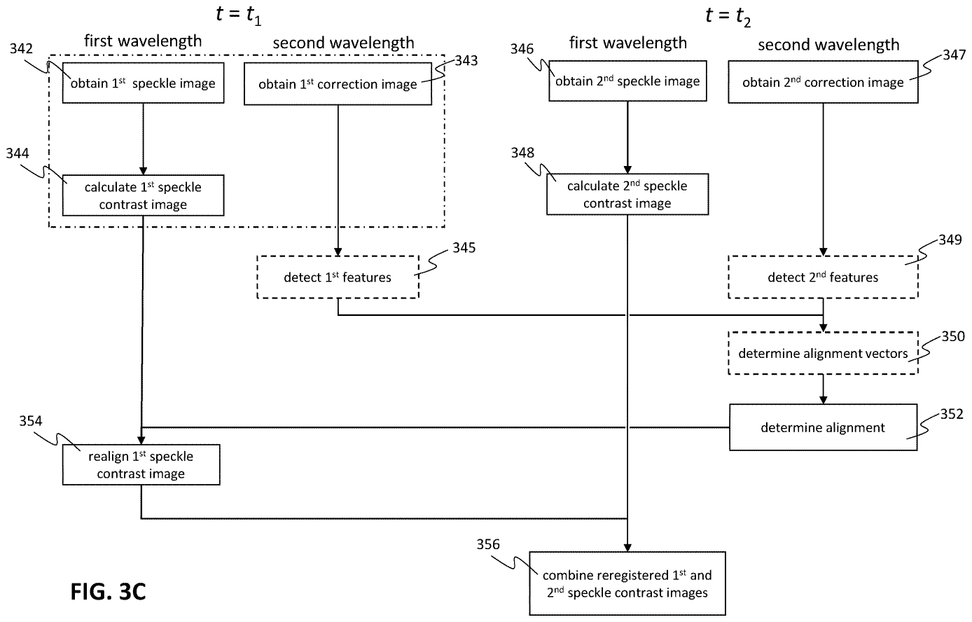

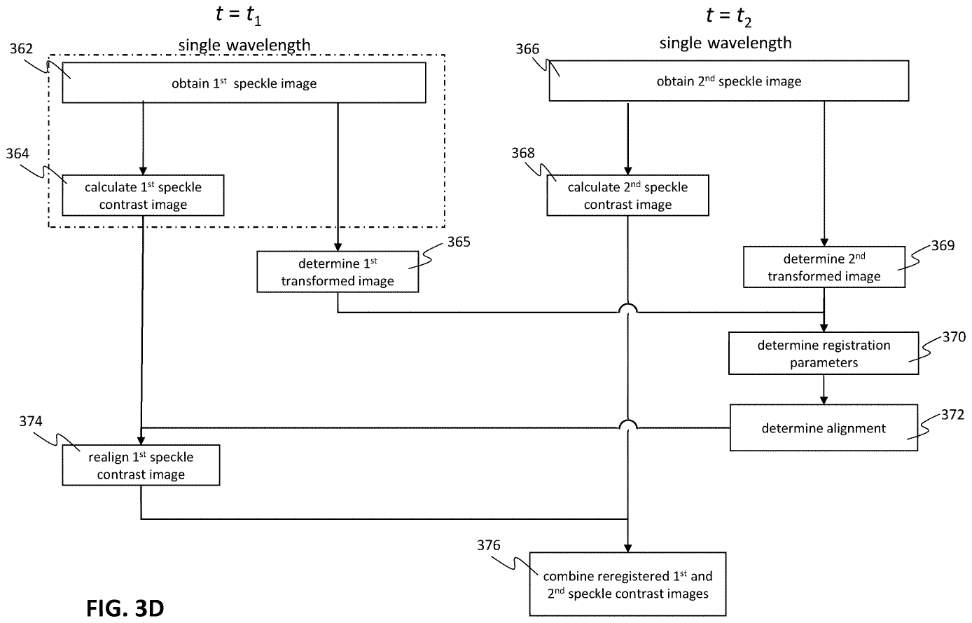

FIG. 3A depicts a flow diagram for motion-compensated laser speckle contrast imaging according to an embodiment. In a first step 302, a first raw speckle image may be obtained at a first time instance t=t₁. A light source may illuminate a target area with coherent light of a predetermined wavelength and an image sensor may capture a first raw speckle image based on the predetermined wavelength. Based on the first raw speckle image, a first laser speckle contrast image may be computed 304. A laser speckle contrast image may be determined, for example, by determining a relative standard deviation of pixel values in a sliding window, e.g. a 3×3 window, a 5×5 window, or a 7×7 window. Generally, a (2n+1)×(2n+1) window may be selected for a natural number n, depending on the speckle size. Alternatively, a convolution with a kernel may be used, where the size of the kernel may be selected based on the speckle size. A relative standard deviation may be determined by computing the standard deviation of pixel intensity values in an area divided by the mean pixel intensity value in the area. Alternatively, laser speckle contrast values may be determined in any other suitable way.

Steps 306 and 308 are analogous to steps 302 and 304, respectively, executed at a second time instance t=t₂. Thus, a second raw speckle image may be obtained 306 at the second time instance t=t₂. The light source may illuminate the target area with coherent light of the predetermined wavelength and the image sensor may capture a second raw speckle image based on the predetermined wavelength. Based on the second raw speckle image, a second laser speckle contrast image may be computed 308.

In a next step 310, the processor may determine registration parameters based on the first and second speckle images, based on images associated with the first and second speckle images, and/or on transformations of the first and second speckle images. The registration parameters describe a geometric relation between the first and second speckle images allowing to align the first and second speckle images with each other. For example, the registration parameters may comprise alignment vectors describing a displacement of one or more pixels in a real or transformed space.

In a next step 312, the processor may determine an alignment transformation, e.g. an affine transformation or a more general homography, for registering or aligning the first and second speckle images with each other, based on the registration parameters. Based on the alignment transformation, the processor may register or align 314 the first laser speckle contrast image and the second laser speckle contrast image with each other, by transforming the first and/or second laser speckle contrast images. Typically, the older image may be transformed to be registered or aligned with the newer image.

The processor may then compute 316 a combined, e.g. averaged, laser speckle contrast image based on the registered first and second laser speckle contrast images. Computing a combined image may comprise computing a weighted average, the weights preferably being based on a normalised amount of speckle contrast, on a relative change in speckle contrast, and/or on the determined registration parameters. Computing a combined image may further comprise applying one or more filters, e.g. a median filter, or an outlier filter.

In other embodiments, the steps may be performed in a different order. For example, the laser speckle contrast images may be computed after the raw speckle images have been registered. This way, temporal or spatio-temporal speckle contrast images may be computed. However, if the alignment transformation is more general than a translation (e.g. comprises rotating, scaling and/or shearing), the alignment transformation may distort the speckle pattern and thus introduce a source of noise. In some embodiments, a single laser contrast raw speckle image may be computed based on the combined, e.g. averaged, raw speckle images. In this case, the images are preferably registered with sub-pixel accuracy.

FIG. 3B depicts a flow diagram for motion-compensated laser speckle contrast imaging according to an embodiment. In a first step 322, a first raw speckle image may be obtained at a first time instance t=t₁ and based on the first raw speckle image, a first laser speckle contrast image may be computed 324.

In a next step 325, a processor may determine a first plurality of first features in the first raw speckle image. Alternatively, the first plurality of first features may be determined in the first speckle contrast image. Preferably, the image with the most clearly defined anatomical features is used; it may depend on the imaging parameters such as wavelength and exposure time whether the anatomical have a higher visual distinctiveness in the raw speckle image or in the speckle contrast image. In the remaining part of the description of FIG. 3A, the term ‘speckle image’ may refer to either a raw speckle image or a speckle contrast image. The steps relating to feature detection will be described in more detail below with reference to FIGS. 5A and 6A-D.

Steps 326-329 are analogous to steps 322-325, respectively, executed at a second time instance t=t₂. Thus, a second raw speckle image may be obtained 326 at the second time instance t=t₂. The light source may illuminate the target area with coherent light of the predetermined wavelength and the image sensor may capture a second raw speckle image based on the predetermined wavelength. Based on the second raw speckle image, a second laser speckle contrast image may be computed 328.

In a next step 329, the processor may determine a second plurality of second features in the second speckle image. At least a part of the second plurality of second features should correspond to at least a part of the first plurality of first features. Typically, the second speckle image is similar to the first speckle image, as in a typical application, the target area will not change much between t=t₁ and t=t₂. Therefore, when a deterministic algorithm is used to detect features, most of the features detected in the second speckle image will generally correspond to features detected in the first speckle image, in the sense that the detected features in the images represent the same, or practically the same, anatomical features in the imaged target. Thus, a plurality of second features may be associated with a plurality of first features.

In a next step 330, the processor may determine a plurality of alignment vectors based on the first features and the corresponding second features, a alignment vector describing the displacement of a feature relative to an image. For example, the processor may determine pairs of features comprising one first feature and one second feature, determine a first position of the first feature relative to the first speckle image, determine a second position of the second feature relative to the second speckle image, and determine a difference between the first and second positions. Typically, pairs of corresponding features may be pairs of a first feature and an associated second feature representing the same anatomical feature.

In a next step 332, the processor may determine a transformation, e.g. an affine transformation or a more general homography, for registering corresponding features with each other, based on the plurality of alignment vectors. The transformation may e.g. be found by selection from a class of transformations a transformation that minimizes a distance between pairs of corresponding features. Based on the transformation, the processor may register or align 334 the first laser speckle contrast image and the second laser speckle contrast image with each other, by transforming the first and/or second laser speckle contrast images. Typically, the older image may be transformed to be registered with the newer image.

In other embodiments, steps 325 and 329 may be omitted, and alignment vectors may be determined based on the first and second speckle images. For example, a dense optical flow algorithm may be used, such as a Pyramid Lucas-Kanade algorithm or a Farneback algorithm to determine alignment vectors. This sort of algorithms typically performs a convolution of a pixel neighbourhood from the first speckle image with a part or the whole of the second speckle image, thus matching a neighbourhood for each pixel in the first speckle image with a neighbourhood in the second speckle image. Such methods may also comprise determining a polynomial expansion to model pixel values in pixel neighbourhoods in the first and second speckle images, comparing expansion coefficients, and determining alignment vectors based on the comparison.

This way, alignment vectors may be determined for e.g. individual pixels or pixel groups, based on pixel values in pixel groups in the speckle images.

In some embodiments, step 330 may be omitted, and a transformation may be determined based on pixel values of pixel groups in the first speckle image and pixel values of associated pixel groups in the second speckle image, for instance using a trained neural network that receives a first image and a second image as input and provides as output a transformation to register the first image with the second image or, alternatively, the second image with the first image.

The processor may then compute 336 a combined, e.g. averaged, laser speckle contrast image based on the registered first and second laser speckle contrast images. Computing a combined image may comprise computing a weighted average, the weights preferably being based on a normalised amount of speckle contrast, on a relative change in speckle contrast, on the determined alignment vectors, and/or on parameters associated with the determined transformation. Computing a combined image may further comprise applying one or more filters, e.g. a median filter, or an outlier filter.

FIG. 3C depicts a flow diagram for motion-compensated laser speckle contrast imaging according to an embodiment. In a first step 342, a first raw speckle image may be obtained at a first time instance t=t₁. A first light source may illuminate a target area with coherent light of a first wavelength and a first image sensor may capture a first raw speckle image based on the first wavelength. Based on the first raw speckle image, a first laser speckle contrast image may be computed 344. A laser speckle contrast image may be determined as described above with reference to step 304.

In a next step 343, a first correction image associated with the first speckle image may be obtained at a first time instance t=t₁. The first correction image may comprise pixels with pixel values and pixel coordinates, the pixel coordinates identifying the position of the pixel in the image. The first correction image is preferably obtained simultaneously with the first raw speckle image, but in an alternative embodiment, the first correction image may be obtained e.g. before or after the first raw speckle image.

A second light source may illuminate the target area with light of at least a second wavelength, different from the first wavelength and a second image sensor may capture a first correction image based on the at least second wavelength. The second light source may use coherent light or incoherent light. The second light source may generate monochromatic light or polychromatic light, e.g. white light. The second image sensor may be the same sensor as the first image sensor, or a different sensor.

In the embodiment described above with reference to FIG. 3A, the second wavelength is the same as the first wavelength, and the first correction image is the same image as the first raw speckle image.

In a next step 345, a processor may determine a first plurality of first features in the first correction image. The steps relating to feature detection will be described in more detail with reference to FIG. 5-6.

Steps 346-349 are analogous to steps 342-345, respectively, executed at a second time instance t=t₂. Thus, a second raw speckle image may be obtained 346 at the second time instance t=t₂. The first light source may illuminate the target area with coherent light of the first wavelength and the first image sensor may capture a second raw speckle image based on the first wavelength. Based on the second raw speckle image, a second laser speckle contrast image may be computed 348.

In a next step 345, a second correction image associated with the second raw speckle image may be obtained at the second time instance t=t₂. The second light source may illuminate the target area with light of the at least second wavelength and the second image sensor may capture a second correction image based on the at least second wavelength.

In a next step 349, the processor may determine a second plurality of second features in the second correction image. At least a part of the second plurality of second features should correspond to at least a part of the first plurality of first features. Typically, the second correction image is similar to the first correction image, as in a typical application, the target area will not change much between t=t₁ and t=t₂. Therefore, when a deterministic algorithm is used to detect features, most of the features detected in the second correction image will generally correspond to features detected in the first correction image, in the sense that the detected features in the images represent the same, or practically the same, anatomical features in the imaged target. Thus, a plurality of second features may be associated with a plurality of first features.

In a next step 350, the processor may determine a plurality of alignment vectors based on the first features and the corresponding second features, a alignment vector describing the displacement of a feature relative to an image. For example, the processor may determine pairs of features comprising one first feature and one second feature, determine a first position of the first feature relative to the first correction image, determine a second position of the second feature relative to the second correction image, and determine a difference between the first and second positions. Typically, pairs of corresponding features may be pairs of a first feature and an associated second feature representing the same anatomical feature.

In a next step 352, the processor may determine an alignment transformation, e.g. an affine transformation or a more general homography, for registering corresponding features with each other, based on the plurality of alignment vectors. The alignment transformation may e.g. be found by selection from a class of transformations a transformation that minimizes a distance between pairs of corresponding features. Based on the alignment transformation, the processor may register or align 354 the first laser speckle contrast image and the second laser speckle contrast image with each other, by transforming the first and/or second laser speckle contrast images. Typically, the older image may be transformed to be registered with the newer image. In embodiments with more than one image sensor, the registration parameters determined based on the correction images may be adjusted to account for differences between the fields of view of the more than one image sensor.

In other embodiments, steps 345 and 349 may be omitted, and alignment vectors may be determined based on the first and second correction images. For example, a dense optical flow algorithm may be used, such as a Pyramid Lucas-Kanade algorithm or a Farneback algorithm to determine alignment vectors. This sort of algorithms typically performs a convolution of a pixel neighbourhood from the first correction image with a part or the whole of the second correction image, thus matching a neighbourhood for each pixel in the first correction image with a neighbourhood in the second correction image. Such methods may also comprise determining a polynomial expansion to model pixel values in pixel neighbourhoods in the first and second correction images, comparing expansion coefficients, and determining alignment vectors based on the comparison.

This way, alignment vectors may be determined for e.g. individual pixels or pixel groups, based on pixel values in pixel groups in the first correction image and associated pixel groups in the second correction image.

In some embodiments, step 350 may be omitted, and the alignment transformation may be determined based on pixel values of pixel groups in the first correction image and pixel values of associated pixel groups in the second correction image, for instance using a trained neural network that receives a first image and a second image as input and provides as output a transformation to register the first image with the second image or alternatively, the second image with the first image.

The processor may then compute 356 a combined, e.g. averaged, laser speckle contrast image based on the registered first and second laser speckle contrast images, as described above with reference to step 316.

In other embodiments, the steps may be performed in a different order. For example, the laser speckle contrast images may be computed after the raw speckle images have been registered. This way, temporal or spatio-temporal speckle contrast images may be computed. However, if the transformation is more general than translation and rotation (e.g. comprises scaling or shearing), the transformation may distort the speckle pattern and thus introduce a source of noise. This is particularly true for embodiments with more than one camera. In some embodiments, a single laser contrast raw speckle image may be computed based on the combined, e.g. averaged, raw speckle images. In this case, the images are preferably registered with sub-pixel accuracy.

FIG. 3D depicts a flow diagram for motion-compensated laser speckle contrast imaging according to an embodiment. In a first step 362, a first raw speckle image may be obtained at a first time instance t=t₁ and based on the first raw speckle image, a first laser speckle contrast image may be computed 364.

In a next step 365, a processor may determine a first transformed image based on the first raw speckle image or based on the first speckle contrast image, for example a Fourier transform, a Mellin transform, or a log-polar coordinate transform. The steps relating to image transformation will be described in more detail below with reference to FIGS. 5C-F.

Steps 366-369 are analogous to steps 362-365, respectively, executed at a second time instance t=t₂. Thus, a second raw speckle image may be obtained 366 at the second time instance t=t₂. The light source may illuminate the target area with coherent light of the predetermined wavelength and the image sensor may capture a second raw speckle image based on the predetermined wavelength. Based on the second raw speckle image, a second laser speckle contrast image may be computed 368. A second transformed image may be determined 369 based on the second speckle image, using the same transformation as in step 365.

In a next step 370, the processor may determine registration parameters based on the first and second speckle images, based on images associated with the first and second speckle images, and/or on transformations of the first and second speckle images. The registration parameters describe a geometric relation between the first and second speckle images allowing to align the first and second speckle images with each other. For example, the registration parameters may comprise alignment vectors describing a displacement of one or more pixels in a real or transformed space.

In a next step 372, the processor may determine an alignment transformation, e.g., an affine transformation or a more general homography, for registering or aligning the first and second speckle images with each other, based on the registration parameters. Based on the alignment transformation, the processor may register or align 374 the first laser speckle contrast image and the second laser speckle contrast image with each other, by transforming the first and/or second laser speckle contrast images. Typically, the older image may be transformed to be registered or aligned with the newer image.

The processor may then compute 376 a combined, e.g. averaged, laser speckle contrast image based on the registered first and second laser speckle contrast images. Computing a combined image may comprise computing a weighted average, the weights preferably being based on a normalised amount of speckle contrast, on a relative change in speckle contrast, on the determined alignment vectors, and/or on parameters associated with the determined transformation. Computing a combined image may further comprise applying one or more filters, e.g. a median filter, or an outlier filter.

The examples described above with reference to FIG. 3A-D may be combined. For example, the registration parameters may comprise translation parameters, rotation parameters, and scaling parameters, where the translation parameters are determined based on the speckle images, e.g. using template matching, while the rotation parameters and scaling parameters are determined based on the transformed images, e.g. using cross-correlation of log-polar images.

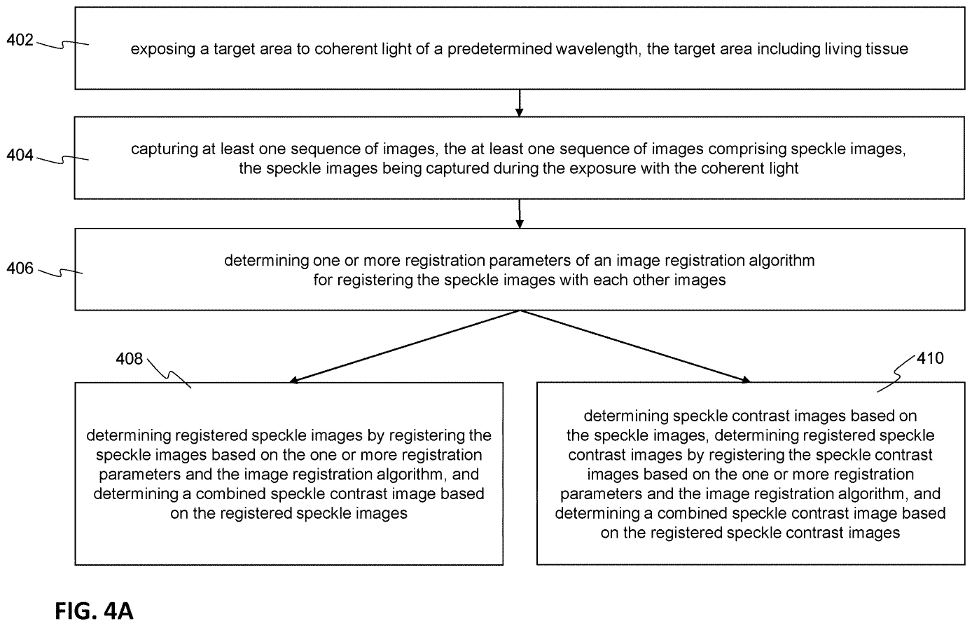

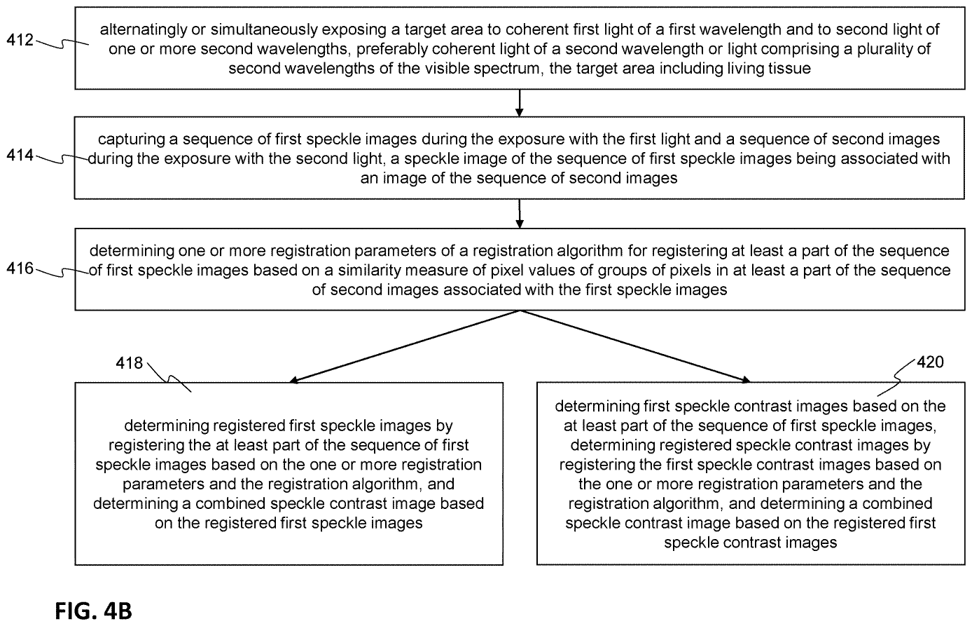

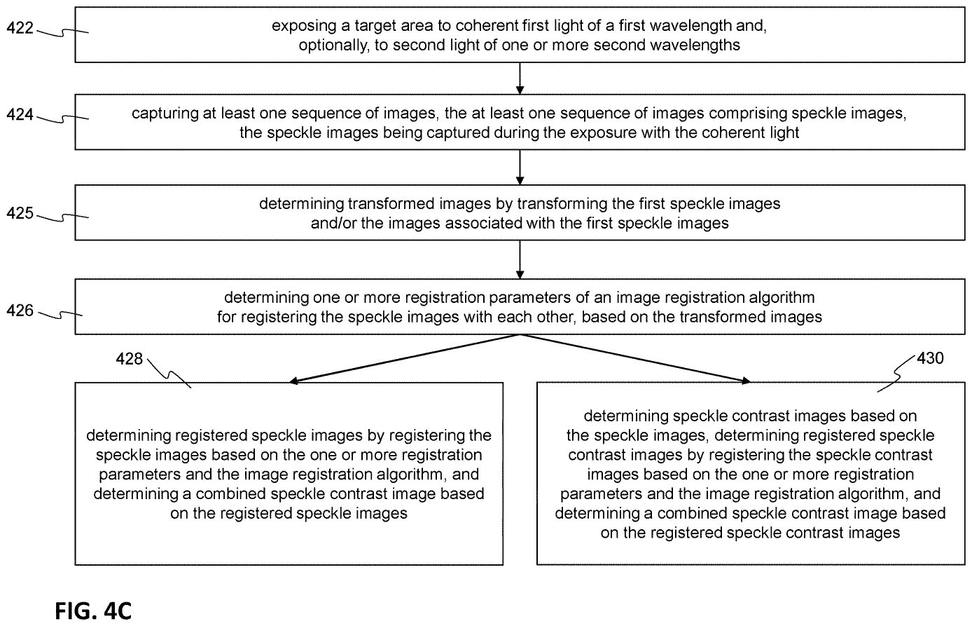

FIG. 4A depicts a flow diagram for a motion-compensated speckle contrast imaging method according to an embodiment. In a first step 402, the method may comprise exposing a target area to coherent light of a predetermined wavelength. Preferably, the target area comprises living tissue, e.g. skin, burns, or internal organs such as intestines, or brain tissue. Preferably, the living tissue is perfused and/or comprises blood vessels and/or lymph vessels. The predetermined wavelength may be a wavelength in the visible spectrum, e.g. in the red, green, or blue part of the visible spectrum, or the predetermined wavelength may be a wavelength in the infrared part of the spectrum, preferably in the near-infrared part.

In a next step 404, the method may comprise capturing, e.g. by an image sensor, at least one sequence of images, the at least one sequence of images comprising (raw) speckle images, the (raw) speckle images being captured during the exposure with the first light.

Each raw speckle image may comprise pixels, the pixels being defined by pixel coordinates and having pixel values. The pixel coordinates may define the position of the pixel relative to the image, and are typically associated with a sensor element of the image sensor. The pixel value may represent a light intensity.

The image sensor may comprise a 2D image sensor, e.g. a CCD, for example a monochrome camera or a colour camera. The images in the sequence of images can be frames in a video stream or in a multi-frame snapshot.

In a next step 406, the method may further comprise determining one or more registration parameters of an image registration algorithm for registering the speckle images with each other. In principle, the registration parameters may be determined in any suitable, for example as described below with reference to FIGS. 5A-F and 6A-D.

For example, the registration parameters may be based on a similarity measure of pixel values of pixel groups in a plurality of images of the at least one sequence of images, the images in the plurality of images being selected from the speckle images. Alternatively, the images in the plurality of images may comprise other images than the raw speckle images, that are associated with the raw speckle images. The registration parameters preferably define one or more transformations out the group of homographies, projective transformations, or affine transformations.

The determination of the registration parameters based on pixel groups is described in more detail below with reference to step 416, with the understanding that in this embodiment, the (raw) speckle images are used as the correction images.

The method may further comprise determining a combined laser speckle contrast image based on the at least part of the sequence of first speckle images and the one or more determined registration parameters, using either step 408 or step 410.

In a step 408, the method may further comprise determining registered speckle images by registering the speckle images based on the one or more registration parameters and the image registration algorithm, and determining a combined speckle contrast image based on the registered speckle images. In such an embodiment, the algorithm may first register the sequence of raw speckle images using the determined transformation, then compute a sequence of speckle contrast images, and then combine the registered speckle contrast images.

In an alternative step 410, the method may further comprise determining speckle contrast images based on the speckle images, determining registered speckle contrast images by registering the speckle contrast images based on the one or more registration parameters and the image registration algorithm, and determining a combined speckle contrast image based on the registered speckle contrast images. In other words, the algorithm may first compute a sequence of speckle contrast images, then register the speckle contrast images using the determined transformation, and then combine the registered speckle contrast images.

Further alternatives are discussed below with reference to steps 418 and 420.

FIG. 4B depicts a flow diagram for a motion-compensated speckle contrast imaging method according to an embodiment. In a first step 412, the method may comprise alternatingly or simultaneously exposing a target area to coherent first light of a first wavelength and to second light of one or more second wavelengths, preferably at least in part different from the first wavelength. The second light may be, for example, coherent light of a second wavelength, narrow-band light, or light comprising a plurality of second wavelengths of the visible spectrum, e.g. white light. Preferably, the target area comprises living tissue, e.g. skin, burns, or internal organs such as intestines, or brain tissue. Preferably, the living tissue is perfused and/or comprises blood vessels and/or lymph vessels

In a next step 414, the method may comprise capturing, e.g. by an image sensor system with one or more image sensors with a fixed relation to each other, a sequence of first (raw) speckle images during the exposure with the first light, and a sequence of second (correction) images during the exposure with the second light. A speckle image of the sequence of first speckle images may be associated with an image of the sequence of second images.

In the case of simultaneous exposure and acquisition, each second image may be associated with the simultaneously acquired first speckle image. In an embodiment where the second images are the same images as the first speckle images, each image may be considered associated with itself, and the image may be referred to as a first speckle image or as a second image, depending on its function in the algorithm (e.g. determining registration parameters or providing perfusion information).

In the case of alternating acquisition, a first speckle image may be associated with, e.g., the second image acquired immediately preceding or subsequent to the first speckle image, or both. When the first speckle images are acquired at a higher rate than the second images, several first speckle images may be associated with a single second image. Thus, a sequence of first raw speckle images of the target area and a sequence of correction images of the target area may be acquired, each correction image being associated with one or more first raw speckle images. Each correction image may comprise pixels, the pixels being defined by pixel coordinates and having pixel values. The pixel coordinates may define the position of the pixel relative to the image, and are typically associated with a sensor element of the image sensor. The pixel value may represent a light intensity.

The image sensor system may comprise one or more 2D image sensors, e.g. CCDs. The first raw speckle images and the correction images may be acquired using one or more image sensors, for example using greyscale cameras or colour (RGB) cameras. The images in the sequence of images may be e.g. frames in a video stream or multi-frame snapshot.

In a next step 416, the method may further comprise determining one or more registration parameters of a registration algorithm for registering at least a part of the sequence of first speckle images based on a similarity measure of pixel values of pixel groups in at least a part of the sequence of second images associated with the first speckle images. The registration parameters preferably define one or more transformations out the group of homographies, projective transformations, or affine transformations.

Determining registration parameters may comprise selecting a first correction image from the at least part of the sequence of correction images and determining a plurality of first pixel groups in the first correction image. The first correction image may be a reference correction image. In an embodiment, the first correction image may be the first image, e.g. when a single output image is generated based on input by a user. In a different embodiment, the first correction image may be the most recent correction image, e.g. when a continuous stream of output images is being generated.

A first pixel group may be associated with a feature in the first correction image, e.g. an edge or corner. Preferably, a feature is associated with a physical or anatomical feature, e.g. a blood vessel, or more in particular, a sharp corner or a bifurcation in a blood vessel. Image features not related to physical features, such as overexposed image parts or edges of speckles, may display a large inter-frame variation, and may hence be less useful to register images. The features may be predetermined features, e.g. features belonging to a class of features, such as corners or regions with large differences in intensity. Features may further be determined by e.g. a quality metric, restrictions on mutual distances between features, et cetera.

Alternatively, a pixel group may be associated with a region in the first correction image, e.g. a neighbourhood of a predetermined set of pixels. Thus, the pixel group may comprise, for example, every pixel in the image, a contiguous region of pixels at a predetermined location, e.g., the centre of the image, or a selection of pixels equally distributed over the image.

Determining registration parameters may further comprise selecting one or more second correction images, different from the first correction image. For each of the selected one or more second correction images, a plurality of second pixel groups may be determined. The second pixel groups may be associated with a feature in the second correction image. If feature-based (image) registration is used, e.g. using a sparse optical flow algorithm, preferably, the same algorithm is used to determine the first pixel groups and the second pixel groups.

If the first pixel groups are determined based on pixel coordinates, the second pixel groups may be determined by convolving or cross-correlating a first pixel group with the second correction image and e.g. selecting the pixel group that is most similar to the first pixel group, based on a suitable similarity metric. The convolution may be restricted in space, e.g. by only searching for a matching second pixel group close to the position of the first pixel group. Alternatively or additionally, the second pixel groups may be restrained to conserve the mutual orientation of the first pixel groups, e.g. for preventing anatomically impossible combinations.

The second pixel groups may then be associated with the first pixel groups based on, at least, a similarity in pixel values. In an embodiment where the second pixel groups are determined by matching or convolution with the first pixel groups, such association may be performed as part of determining the second pixel groups. If feature-based registration is used, a second pixel group may be associated with a first pixel group based on similarity of the feature associated with the second pixel group and the feature associated with the first pixel group.

A transformation for registering the second correction image and the first correction image, and hence for registering the associated first speckle images or derived first speckle contrast images, may be determined based on the pixel coordinates of pixels in the associated first and second pixel groups. Determining a transformation may comprise determining a 3D motion of the image sensor system relative to the target area, or may be informed by the effects this 3D motion would have on the acquired images.

As an intermediate step, alignment vectors may be determined, based on positions of the first and associated second pixel groups, e.g. based on positions of features in the first and second correction images. The alignment vectors may represent motion of the target area relative to the image sensor or image capturing device. In some embodiments, the neighbourhood of one or more determined object features may be used to determine the alignment vectors and/or the transformation.

The determination of alignment vectors and/or the determination of the transformation may be based on optical flow parameters, determined using any suitable sparse or dense optical flow algorithm. Determining alignment vectors may comprise determining pairs of corresponding or matching features, one feature of a pair of features being determined in a correction image associated with a first time instance, the other feature in the pair of features being determined in the subsequent correction image in the sequence of correction images. associated with a subsequent time instance.

Methods to determine registration parameters and, optionally, features, are discussed in more detail below with reference to FIGS. 5 and 6.

The method may further comprise determining a combined laser speckle contrast image based on the at least part of the sequence of first speckle images and the one or more determined registration parameters, using either step 418 or step 420.

In a step 418, the method may further comprise determining registered first speckle images by registering the at least part of the sequence of first speckle images based on the one or more registration parameters and the registration algorithm, and determining a combined speckle contrast image based on the registered first speckle images. In such an embodiment, the algorithm may first register the sequence of raw speckle images using the determined transformation, then compute a sequence of speckle contrast images, and then combine the registered speckle contrast images.

In an alternative step 420, the method may further comprise determining first speckle contrast images based on the at least part of the sequence of first speckle images, determining registered speckle contrast images by registering the first speckle contrast images based on the one or more registration parameters and the registration algorithm, and determining a combined speckle contrast image based on the registered first speckle contrast images. In other words, the algorithm may first compute a sequence of speckle contrast images, then register the speckle contrast images using the determined transformation, and then combine the registered speckle contrast images.

In a further alternative, determining a combined laser speckle contrast image may comprise determining a sequence of registered first raw speckle images based on the first raw speckle images and the determined transformation, determining a combined speckle contrast image based on two or more registered first raw speckle images of the sequence of registered first raw speckle images, and determining a combined speckle contrast image based on the combined raw speckle image. In such an embodiment, the algorithm may first register the sequence of raw speckle images using the determined transformation, then combine the registered speckle contrast images, and then compute a speckle contrast image.

In an even further alternative, the combining and the computing of a speckle contrast may be a single step, e.g. by computing a temporal or spatio-temporal speckle contrast based on the sequence of registered first raw speckle images.

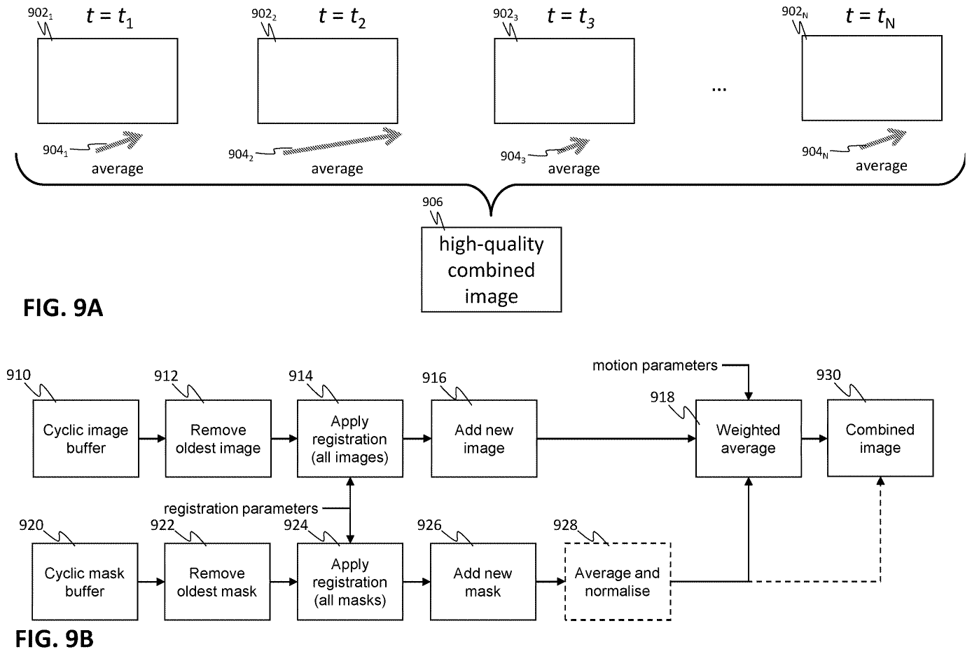

Combining raw speckle images or speckle contrast images may comprise averaging, weighted averaging, filtering with e.g. a median filter, et cetera. Weights for weighted averaging may be based e.g. on a quantity derived from the speckle contrast, derived from the registration parameters, or derived from the alignment vectors. Methods of combining raw speckle images or speckle contrast images are discussed in more detail below with reference to FIG. 9A.

FIG. 4C depicts a flow diagram for a motion-compensated speckle contrast imaging method according to an embodiment. Step 422 and 424 may be analogous to steps 402 and 404 or steps 412 and 414, respectively.

In a next step 425, the method may further comprise determining transformed images by transforming the first speckle images and/or images associated with the first speckle images (e.g., image captured during exposure with the second light). The transformed images may be obtained using, e.g., a Fourier transform, a Mellin transform, and/or a log-polar coordinate transform.

In a next step 426, the method may further comprise determining one or more registration parameters of an image registration algorithm for registering the speckle images with each other, based on the transformed images. Examples are described below with reference to FIGS. 5D-F and 6.

The method may further comprise determining a combined laser speckle contrast image based on the at least part of the sequence of first speckle images and the one or more determined registration parameters, using either step 428 or step 430. These steps are analogous to steps 408 and 410, respectively steps 418 and 420, discussed above.

FIG. 5A depicts a method for determining registration parameters according to an embodiment. A first plurality of first features 506₁₋₃ may be determined in a first correction image 502, acquired at a first time instance t=t₁. Each of the plurality of first features may be associated with a pixel group in the first correction image. Preferably, the first features relate to anatomical structures, e.g. blood vessels 504₁₋₂, or other stable features that may be assumed not to move between subsequent frames. Therefore, the first correction image is preferably obtained using light which makes such anatomical structures clearly visible. For example, green light may be used, which is strongly absorbed by blood vessels, but not by most other tissues. Therefore, blood vessels may appear as dark structures in a light environment, resulting in a high visual distinctiveness. However, other embodiments may use light of one or more other wavelengths, e.g. blue light or white light.

Features 506₁₋₃ may be determined using any suitable feature detection algorithm, for example a feature detector based on a Harris detector or Shi-Tomasi detector, such as goodFeatures ToTrack from the OpenCV library. Other examples of suitable feature detectors and descriptors include Speeded-Up Robust Features (SURF), Features from Accelerated Segment Test (FAST), Binary Robust Independent Elementary Features (BRIEF), and combinations thereof such as ORB. Various suitable algorithms have been implemented in generally available image processing libraries such as OpenCV. Preferably, depending on the application, the algorithm should be fast enough to allow real-time image processing.

Typically, sharp corners (e.g. features 506_{1,3}) and bifurcations (e.g. feature 506₂) are good features. Preferably, a deterministic feature detection algorithm is used, i.e., a feature detection algorithm that detects identical features in identical images. Preferably, the features should be distributed over a large part of the image area. A good distribution of feature points over the image may be obtained by requiring a minimum distance between selected feature points. In some embodiments, e.g. based on implementations of BRIEF or ORB, the features may be assigned a descriptor identifying feature properties, facilitating feature distinction and feature matching.

A minimum number of feature points depends on the type of transformation; for example an affine transformation has six degrees of freedom, while a homography has eight degrees of freedom. Thus, the first plurality of first features may comprise at least 5 features, preferably at least 25, more preferably at least 250, even more preferably at least 1000. The number of features may depend on the amount of pixels in the image, with a larger number of features being used for images with more pixels. Typically, a higher number of features may result in a more accurate transformation, as random errors may be averaged out.

However, there are various reasons why the number of features may be limited. For example, there may only be a limited number of features that satisfy predetermined quality indicators, e.g. a magnitude of a local contrast or the sharpness of a corner. Additionally, the computation time increases with the number of features, and hence, the number of features may be limited to allow real-time image registration, e.g. for a 50 fps video feed, the entire algorithm should preferably take less than 20 ms per frame.

A second plurality of second features 516₁₋₃ associated with second pixel groups may be determined in a second correction image 512, acquired at a second time instance t=t₂. Preferably, the field of view of the second correction image overlaps substantially, preferably more than half, with the field of view of the first correction image. Preferably, the second features relate to the same anatomical structures, e.g. blood vessels 514₁₋₂ as the first features. Preferably, the same feature detection algorithm is used to detect features in both the first and second correction images.

The determined first and second features may comprise position information relative to the first and second correction images, respectively. In this example, this is shown in comparison image 522. Based on the first plurality of first features 506₁₋₃ and the second plurality of second features 516₁₋₃, a plurality of alignment vectors 524₁₋₂ may be determined, an alignment vector describing the displacement of a feature relative to an image. In an intermediate step, pairs of corresponding features may be determined, e.g. feature 506₁ may be associated with feature 516₁, feature 506₂ may be associated with feature 516₂, and feature 506₃ may be associated with feature 516₃. Pairs of corresponding features may e.g. be determined based on feature parameters such as local contrast or the sharpness of a corner, or based on distances between features in the first and second correction images. In some embodiments, not all features in the first correction image can be paired to features in the second correction image. In some embodiments, determining alignment vectors 524₁₋₃ and determining pairs of corresponding features may be performed in a single step.

For example, if a minimum distance between features is imposed and the displacement is assumed to be smaller than the minimum distance, an algorithm that minimizes the distance between point clouds formed by, respectively, the first and second features, may implicitly determine pairs of corresponding features and alignment vectors for each pair of corresponding features. In a typical embodiment, the typical inter-frame displacement is only a few pixels. In an embodiment, the plurality of alignment vectors may be filtered to exclude potential outliers, e.g. alignment vectors that deviate more than a predetermined amount from alignment vectors originating from nearby features.

In other embodiments, alignment vectors may be determined based on associated pixel groups, based on pixel values in the first and second correction images. As was explained above with reference to FIG. 3, in such embodiments feature detection may be omitted. Instead, alignment vectors may be determined using e.g. a dense optical flow algorithm, such as a Pyramid Lucas-Kanade algorithm or a Farneback algorithm. In principle, any method to determine alignment vectors based on pixel values of corresponding pixel groups may be used.

Based on the plurality of alignment vectors 524₁₋₃, a transformation may be determined. In an embodiment, the transformation may be defined by an average or median alignment vector, or by another alignment vector that is statistically representative of the plurality of alignment vectors. In a different embodiment, the transformation may be an affine transformation, a projective transformation, or a homography, combining e.g. translation, rotation, scaling and shearing transformations. Preferably, the transformation images features from the first correction image onto the corresponding features in the second correction image. The transformation may then be applied to the first raw speckle image to register the first raw speckle image with the second raw speckle image.

In an embodiment, the first and second correction images may be pre-processed before determining features. For example, overexposed and/or underexposed regions may be identified based on pixel values, e.g., as described above with reference to FIG. 1E. Subsequently, these regions may be masked, so no features may be detected in those regions. The mask may be slightly larger than the overexposed or underexposed region, e.g. by growing the identified region with a predetermined number of pixels. Masking overexposed and/or underexposed regions may improve the quality of the features, as it prevents features e.g. associated with an edge or corner of an overexposed region. If a mask has been determined to identify artifacts, as described above with reference to FIG. 1E, the same mask may be applied prior to the feature detection in order to prevent unreliable features from being detected.

FIG. 5B depicts a further method for determining registration parameters according to an embodiment. This method is sometimes referred to as template matching. A first region 531 is selected in a first image 530, acquired at a first time instance t=t₁. The first image can be, e.g., a raw speckle image, a speckle contrast image, or a correction image. The first region has a predetermined size, expressed in pixels, and is selected at a predetermined location in pixel coordinates. The first region is preferably selected at or near a centre of the first image.

A second region 533 is selected in a second image 532, acquired at a second time instance t=t₂. The second image is typically the same sort of image as the first image, e.g., both can be raw speckle images. The second region has a predetermined size, expressed in pixels, and is selected at a predetermined location in pixel coordinates. The second region is typically smaller than the first region. The size difference can be selected based on an estimated or expected amount of motion. The centre of the second region typically coincides with the centre of the first region. Preferably, the second region is selected with respect to the first region in such a way that there is a high probability that the imaged (anatomical) region represented by the second region is contained in the imaged region represented by the first region. A single region in each image may suffice to determine a global translation registration parameter. As will be discussed in more detail below with reference to FIG. 6D, a plurality of first regions may be selected in the first image and a corresponding plurality of second regions may be selected in the second image, e.g., to determine non-global (i.e., regional or local) registration parameters. The first and second regions may be selected in an overlapping or non-overlapping manner.

Based on the first and second regions, registration parameters may be determined. In this example, this is shown in comparison image 534. For example, a sub-region in the first region may be determined which is most similar to the second region; this step is also known as matching. The registration parameters, e.g., an alignment vector 535, may be determined based on the relative positions of the second region and the sub-region in the first region. The matching method can be, for example, feature-based, intensity-based, or frequency-based. An example of feature-based matching has been discussed above with reference to FIG. 5A, and an example of frequency-based matching is discussed below with reference to FIG. 5C. An example of intensity-based matching is cross-correlation of the first and second regions. The matching method may also comprise, e.g., a coordinate transformation as discussed below with reference to FIG. 5D. Depending on the matching method, the registration parameters can comprise translation parameters, rotation parameters, and/or other parameters.

FIG. 5C depicts a further method for determining registration parameters according to an embodiment. FIG. 5C depicts a frequency-based registration algorithm, also known as phase correlation. A first image 540, acquired at a first time instance t=t₁, is transformed using a transformation resulting in a first transformed image 541. In this example, a discrete Fourier transformation is used. Similarly, a second image 542, acquired at a second time instance t=t₂, is transformed using the discrete Fourier transformation, resulting in a second transformed image 543. Based on a comparison 544 of the first and second transformed images, registration parameter may be determined to register 556 the first and second speckle images.

Image registration based on Fourier transformations are an example of frequency-based image registration. These methods are generally well-known in the art. The comparison of the transformed images may comprise the computation of a cross-power spectrum of the transformed images. The computation of the cross-power spectrum may comprise determining a complex conjugate of one of the transformed images and element-wise multiplying it with the other of the transformed images. The comparison may further comprise determining an (inverse) Fourier transforms of the cross-power spectrum and determining a peak in the resulting image, e.g. by application of an argmax function. Using interpolation, the peak may be determined with sub-pixel accuracy. The location of the peak corresponds to a translation of the images to be aligned. In the depicted example, image 544 represents the inverse Fourier transform of the cross-correlation of the first and second transformed images, and the vector between the top-right corner and the peak 545 represents the translation required to align the second image with the first image.

The comparison may comprise further operations to improve the result. For example, a two-dimensional Hanning window may be applied to the first and second images prior to application of the Fourier transform. As another example, a filter, e.g., a blurring filter or interpolation filter, may be applied to the inverse Fourier transform of the cross-correlation of the transformed images to improve peak detection. Various other operations to improve frequency-based image registration are known in the art. For example, to reduce the effect of noise, high frequencies may be filtered out. On the other hand, a high-pass filter may reduce image artifacts caused by image borders. Additionally, in order to prevent matching of the speckles rather than anatomical structures, frequencies corresponding to speckles may be suppressed. Of course, care should be taken not to suppress all frequencies, in particular not those where the anatomical structures in the image region may give a relatively strong signal. This may depend on the tissue type being imaged.

FIG. 5D depicts a further method for determining registration parameters according to an embodiment. In this example, a so-called log-polar coordinate transformation is used. The log-polar coordinate transformation is typically applied in combination with a method for determining a translation, for which any of the methods discussed above with reference to FIG. 5A-C may be used. Thus, it may be considered a pre-processing step for any of those methods. A first image 550, acquired at a first time instance t=t₁, is transformed using a coordinate transformation resulting in a first transformed image 551. The horizontal axis of the transformed image represents an angle φ with respect to the horizontal axis of the first image, and the vertical axis of the transformed image represents a logarithm of the (relative) radial distance log(r) from the centre of the first image. As r should be dimensionless, r is typically expressed in pixel units or relative to rₘₐₓ, where Imax denotes the maximum distance from the origin (in this example, one of the diagonals). That is, if the pixel coordinates in the first image are represented with x and y (along the horizontal and vertical axis, respectively) with the origin chosen in the centre of the image, then

$$\begin{matrix}φ = arc tan 2 ( y , x ) & x = exp ( r ) cos ( φ ) \\ r = log ( sqrt ( {x}^{2} + {y}^{2} ) ) & y = exp ( r ) sin ( φ )\end{matrix}$$

where arctan 2 (y, x)=arctan (y/x) if x>0, arctan 2 (y, x)=arctan (y/x)+sign (y)*π if x<0 and arctan 2 (y, x)=sign (y)*π/2 if x=0. Other embodiments may use different coordinate transformations.

Similarly, a second image 552, acquired at a second time instance t=t₂, is transformed using the same coordinate transformation, resulting in a second transformed image 553. In the depicted example, the complete first and second speckle images have been transformed. In other embodiments, only one or more regions of the first and second speckle images are transformed. Usually, at least one region contains the centre of the image.

Based on a comparison 554 of the first and second transformed images, a displacement or shift in the transformed (in this case, log-polar) coordinate system may be determined. The displacement may be determined using any suitable method, e.g., one of the methods as described above with reference to FIG. 5A-C. Based on the determined displacement, registration parameters may be determined to register the first and/or second speckle images with each other 556. In the depicted example, the second image has undergone a small translation in addition to a rotation and scaling, and as a consequence, for small r (the bottom part of the image), the shift (and hence, rotation) appears much larger than it actually is. For large r (the top part of the image), there are no data for all values of r. Therefore, it can be advantageous to use only a limit range of r-values.

An advantage of using a log-polar coordinate transformation is that a horizontal shift 555 of the transformed image corresponds to a rotation 557 of the untransformed image, and that a vertical shift of the transformed image corresponds to a scaling of the untransformed image. Thus, rotation and scaling can be determined in a relatively straightforward way, in particular global scaling and rotation. As, in this method, relatively large image regions are used to determine (global) scaling and rotation registration parameters, a relatively large amount of image data may be used, which can reduce the effect of noise on the determined registration parameters.

Using non-feature-based methods, e.g., as described with reference to FIG. 5B-D, there is generally no need to determine a plurality of alignment vectors, as may be done using feature-based methods as described with reference to FIG. 5A. Instead, for example, a single matrix may be determined based on the registration parameters, which, when applied to the first image, aligns the first image with the second image. This may reduce computational requirements.

FIGS. 5E and 5F depict further methods for determining registration parameters according to an embodiment. In these examples, two or more of the methods described with reference to FIG. 5A-D are combined. For example, the template-matching method described with reference to FIG. 5B may be used to determine translations, while the log-polar coordinate transformation is used to determine rotations and scaling. When several methods are combined, an intermediate registration step may applied between two of the registration parameter determinations. This tends to improve the outcome of the image registration.

FIG. 5E depicts an example wherein, first, translation parameters are determined and applied to first (speckle) image in an intermediate registration step. Subsequently, rotation and scaling parameters are determined and applied, based on the result of the intermediate scaling step. FIG. 5F depicts an example wherein first rotation and scaling parameters are determined and applied, and subsequently translation parameters In general, it is advantageous to determine and apply the largest transformation first.

In particular, FIG. 5E depicts an example wherein a first (correction) image 560 and a second (correction) image 562 are obtained. In a step 563, a translation is determined based on the first and second images, and applied to the first image, resulting in a translated first image 564. The translation may be determined using any suitable method, e.g., as described above with reference to FIG. 5A-C. For example, template matching may be used where the templates are matched using frequency-based phase correlation or intensity-based cross-correlation.

Subsequently, both the translated first image and the second image are transformed 565,567 using a coordinate transformation, in this example a log-polar coordinate transformation (e.g., as described above with reference to FIG. 5D), resulting in a log-polar translated first image 566 and a log-polar second image 568. In a step 569, a shift or displacement is determined based on the log-polar translated first image and the log-polar second image. This shift may be determined using any suitable method, e.g., using one of the methods to determine a translation as described above with reference to FIG. 5A-C. The same method as in step 563 may be used, or a different method. Based on the determined shift, rotation and/or scaling parameters are determined 571 and applied to the translated first image, resulting in a rotated and/or scaled translated first image 572.

The rotated and/or scaled translated first image may then be combined with the second image, resulting in a combined image 574, for example, using a weighted average. As will be discussed in more detail below with reference to FIG. 9A, the weights may be based on the registration parameters. In this example, the registration parameters comprise both translation parameters and rotation and/or scaling parameters. The weights may thus be based on only the translation parameters, only the rotation and/or scaling parameters, or both. For example, a pixel-wise displacement vector may be determined based on the combined registration parameters, and a weight may be based on a statistical representation of the pixel-wise displacement vectors, e.g., an average or a maximum of a norm of the displacement vectors, for all pixels or a subset thereof (e.g., a region of interest).

FIG. 5F depicts a variation of FIG. 5E. A first image 580 and a second (correction) image 582 are obtained. The first and second image are transformed 583,585 using a coordinate transformation, in this example a log-polar coordinate transformation, resulting in a log-polar first image 584 and a log-polar second image 586. In a step 587, a shift is determined based on the log-polar first and second images. Based on the determined shift, rotation and/or scaling parameters are determined 589 and applied to the first image, resulting in a rotated and/or scaled first image 590.

In a step 591, a translation is determined based on the rotated and/or scaled first image and the second image, and the determined translation is applied to the rotated and/or scaled first image, resulting in a rotated and/or scaled translated first image 592. The rotated and/or scaled translated first image may then be combined with the second image, resulting in a combined image 594.

In yet another example, the determination of the rotation and/or scaling and the determination of the translation are both based on the (untransformed) first and second images. An advantage is that in that case, both (sets of) registration parameters may be determined in parallel.

FIG. 6A displays an example of determining registration parameters according to an embodiment, where the determined transformation is a translation. As was explained with reference to FIG. 5A, a first plurality of first features 6021-3 may be determined in a first correction image acquired at t=t₁, and a second plurality of second features 6041-3 may be determined in a second correction image acquired at t=t₂. Based on corresponding pairs of features, a plurality of alignment vectors 6061-3 may be determined. For the sake of clarity, only the features and the alignment vectors are shown, and not the (anatomical) structures.

In a typical situation, the determined alignment vectors 6061-3 will not all be exactly the same. In the depicted example, alignment vector 6061 is slightly shorter than average, while alignment vector 6063 is slightly larger than average. Similarly, the directions of the alignment vector display some variation. Based on the alignment vectors, an average alignment vector 608 may be determined. A translation may be defined by a single vector. For example, all pixels of the first raw speckle image acquired at t=t may be shifted with an amount equal to the average alignment vector. In principle, a translation may be determined based on a single alignment vector. However, by determining a plurality of alignment vectors, the accuracy of the transformation may be improved.

In an embodiment, a similarity between the average alignment vector 608 and the determined alignment vectors 6061-3 may be computed, for example based on the variation of the alignment vectors. This way, an indication may be obtained how well the transformation compensates for the detected displacement of individual pairs of features. Alternatively, the average distance between the features in the first correction image after transformation and the corresponding features in the second correction image may be determined.

FIG. 6B displays an example of determining registration parameters according to an embodiment, where the determined transformation is an affine transformation. Similar to FIG. 6A, a first plurality of first features 612₁₋₃, a second plurality of second features 614₁₋₃, and a plurality of alignment vectors 616₁₋₃ may be determined. In this example, however, the average alignment vector 618, which has almost zero length, is not representative for the determined alignment vectors, which are typically longer, and point in different directions.

Hence, to compensate for this kind of motion, a more general transformation is needed, for example an affine transformation. Affine transformations include translations, rotations, mirroring, scaling, and shearing transformations, and combinations thereof. It is possible to selectively exclude transformations by restricting transformation parameter values. For example, mirroring may be excluded as a possible transformation, as mirroring is typically not physically possible.

In general, an affine transformation can be computed using a transformation matrix with six degrees of freedom as described in equation (1), acting on a point represented in homogeneous coordinates. By restricting the potential values of the affine transformation matrix, the affine transformation may be limited to only predefined operations. For example, a more specific transformation matrix limited tot e.g. rotation matrices can be obtained which can be more suitable for certain applications.

$$[ \begin{matrix}{x}^{′} \\ {y}^{′} \\ 1\end{matrix} ] [ \begin{matrix}{a}_{11} & {a}_{12} & {t}_{x} \\ {a}_{21} & {a}_{22} & {t}_{y} \\ 0 & 0 & 1\end{matrix} ] [ \begin{matrix}x \\ y \\ 1\end{matrix} ] ↔ {p}^{′} = Ap \qquad ( 1 )$$

Here, A is a transformation matrix transforming a point p with coordinates x and y, typically in pixel coordinates, into a transformed point p′ with coordinates x′ and y′. Matrix A comprises six free parameters, of which tₓ and t_{y} define a translation, while the submatrix

$$[ \begin{matrix}{a}_{11} & {a}_{12} \\ {a}_{21} & {a}_{22}\end{matrix} ]$$

may define reflections, rotations, scaling and/or shearing. In this case, submatrix a transformation size may e.g. be based on a norm of the transformation matrix A, or the norm of the matrix A-I, where I is the Identity matrix.

To solve equation (1), at least three alignment vectors may be used to provide a solvable system of six equations and six unknowns. Such a linear system can be solved in a deterministic way as is known in the art. In a typical embodiment, many alignment vectors may be determined, each of which may comprise a small error. Therefore, a more robust approach can be to use multiple alignment vectors and use an appropriate fitting algorithm, e.g., least squares fitting as shown in equation (2).

$$[ A ] = arg ( {min}_{A} ( {\sum }_{i} {‖ {p}_{i}^{′} - {Ap}_{i} ‖}^{2} ) ) \qquad ( 2 )$$

The reliability of the determined transformation may again be determined as was explained above with reference to FIG. 6A.

FIG. 6C displays an example of determining registration parameters according to an embodiment, where the determined transformation is a projective transformation. Projective transformations include and are more general than affine transformations. Projective transformation include e.g. skewing transformations. They may be needed to compensate for e.g. a change in angle between the camera and the target area. Similar to FIGS. 6A and 6B, a first plurality of first features 622₁₋₃, a second plurality of second features 624₁₋₃, and a plurality of alignment vectors 626₁₋₃ may be determined. In this example, the translation on the left side of the image is much smaller than on the right side of the image. Thus, applying an average translation would transform pixels on the left too much, and pixels on the right not enough. This kind of displacement may be corrected by a projective transformation. Various methods to determine a projective transformation based on four or more alignment vectors are known in the art.

In general, a projective transformation can be calculated using a projective matrix as depicted in equation (3) using inhomogeneous coordinates (x₁, y₁, z₁) and (x₂, y₂,

$$[ \begin{matrix}{x}_{2} \\ {y}_{2} \\ {z}_{2}\end{matrix} ] = [ \begin{matrix}{h}_{11} & {h}_{12} & {h}_{13} \\ {h}_{21} & {h}_{22} & {h}_{23} \\ {h}_{31} & {h}_{32} & {h}_{33}\end{matrix} ] [ \begin{matrix}{x}_{1} \\ {y}_{1} \\ {z}_{1}\end{matrix} ] \qquad ( 3 )$$

The inhomogeneous coordinates (x₁, y₁, z₁) may be related to pixel coordinates (x, y) of a feature via x₁=x, y₁=y, and z₁=1, while the transformed pixel coordinates (x′, y′) may be obtained from the inhomogeneous coordinates via x′=x₂/z₂, and y′=y₂/z₂. In some embodiments, alignment vectors may be determined by (x-x, y′-y). In other embodiments, alignment vectors are not explicitly constructed.

Thus, equation (3) may be rewritten as two independent equations as is shown in equation (4).

$${x}^{′} ( {h}_{31} x + {h}_{32} y + {h}_{33} ) = {h}_{11} x + {h}_{12} y + {h}_{13} \qquad ( 4 )$$

$${y}^{′} ( {h}_{31} x + {h}_{32} y + {h}_{33} ) = {h}_{21} x + {h}_{22} y + {h}_{23}$$

Solving for H, equation (4) can be rewritten as equation (5):

$${ν}_{x} h = 0 \qquad ( 5 )$$

$${ν}_{y} h = 0$$

$$where$$

$$h = {( {h}_{11} , {h}_{12} , {h}_{13} , {h}_{21} , {h}_{22} , {h}_{23} , {h}_{31} , {h}_{32} , {h}_{33} )}^{T} \qquad ( 6 )$$

$${ν}_{x} = ( - x , - y , - 1 , 0 , 0 , 0 , {x}^{′} x , {x}^{′} y , {x}^{′} )$$

$${ν}_{y} = ( 0 , 0 , 0 , - x , - y , - 1 , {x}^{′} , y , {x}^{′} )$$

Using a set of N feature points, a system of equations can be setup as shown in equation (7):

$$Vh = 0 \qquad ( 7 )$$

$$where$$

$$V = {( {ν}_{x 1} , {ν}_{y 1} , {ν}_{x 2} , {ν}_{y 2} , … , {ν}_{x N} , {ν}_{y N} )}^{T} \qquad ( 8 )$$

Since by definition the matrix H is homogeneous and can be scaled by any constant, the projective transformation matrix contains eight degrees of freedom. Thus, equation (7) may be solved using only four sets of coordinates provided by four features. The projective transformation matrix H may be normalized using, for example, equation (9):

$${h}_{33} = 1 \qquad ( 9 )$$

or equation (10):

$${h}_{11}^{2} + {h}_{12}^{2} + {h}_{13}^{2} + {h}_{21}^{2} + {h}_{22}^{2} + {h}_{23}^{2} + {h}_{31}^{2} + {h}_{32}^{2} + {h}_{33}^{2} = 0 \qquad ( 10 )$$

Equation (7) can be solved deterministically using at least four points, but using more points may result in a more robust result, similar to what was explained above with regards to the affine transformation. In the case of projective transformations or homographies, this may be done using singular value decomposition (SVD). In some embodiments, the step of determining the alignment vectors or point pairs is combined with the determination of the homography step to find the most accurate projective transformation, this can be done with algorithms such as RANSAC.

In an embodiment, an algorithm may first compute a relatively simple transformation, e.g. a translation. The algorithm may then determine whether the computed transformation reproduces the determined alignment vectors with sufficient accuracy. If not, the algorithm may attempt a more general transformation, e.g. an affine transformation, and repeat the same procedure. This may reduce the required computation time if translations are sufficient in a large enough number of cases. In a different embodiment, the algorithm may always compute a general transformation, e.g. always a general homography. This may result in more accurate registration of the raw speckle images or speckle contrast images.

The examples discussed above with reference to FIG. 6A-C are based on feature-based transformation parameters. However, the same or similar results may be obtained using other methods that provide registration parameters for a plurality of positions in the first and/or second speckle images. For example, the first and/or second speckle images, or transformations thereof, may be divided into a plurality of regions, and registration parameters may be determined for each separate region. The registration parameters for the plurality of regions may be combined as described above with reference to FIG. 6A-C.

FIG. 6D displays an example of determining a plurality of transformations according to an embodiment. Each correction image 650 in the series of correction images may be divided into a plurality of regions 652₁₋ₙ, preferably a plurality of disjoint regions which jointly cover the entire image, for example a rectangular grid. Subsequently, a transformation 654₁, 654₂, . . . , 654ₙ may be determined for each region 654₁, 654₂, . . . , 654ₙ, respectively, for example in the manner as was explained above with reference to FIG. 5A-D or FIG. 6A-C (where the method was applied to the entire image). Subsequently, each region may be transformed using the transformation determined for that region. Alternatively, the determined transformations may be assigned to e.g. a central pixel in the region, and the remaining pixels are transformed according to an interpolation scheme, based on the transformation of the region comprising the pixel and the transformations of neighbouring regions.

In an embodiment, each region may be a single pixel. In such an embodiment, the transformation may be determined based on the pixel value and based on pixel values of pixels in a region surrounding the pixel.

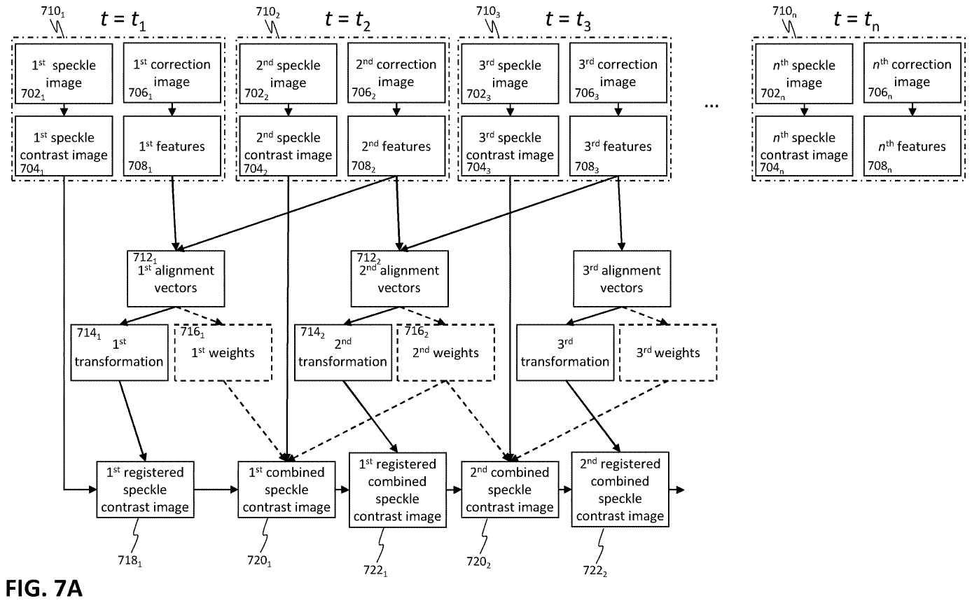

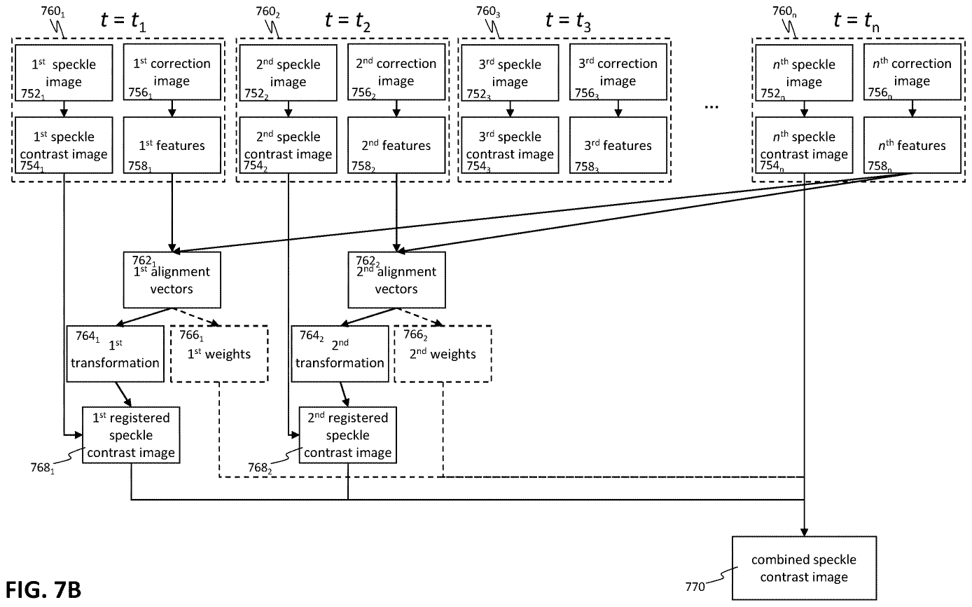

FIGS. 7A and 7B depict flow diagrams for laser speckle contrast imaging combining more than two raw speckle images according to an embodiment. At a first time instance t=t₁, a first raw speckle image 702₁ based on light of a first wavelength may be obtained, and may be used to compute a first laser speckle contrast image 704₁. A first correction image 7061 based on light of at least a second wavelength, and associated with the first raw speckle image may be obtained and a first plurality of first features may be detected 708₁ in the first correction image. Items 702₁-708₁ may be acquired by performing step 710₁, which may be similar to steps 302-308 as described with reference to FIG. 3. In some embodiments, the first correction image and the first raw speckle image may be the same image.

At a second time instance t=t₂, step 710₁ may be repeated as step 710₂, resulting in a second raw speckle image 702₂ based on light of the first wavelength, a second laser speckle contrast image 704₂, a second correction image 706₂ based on light of the at least second wavelength, and a plurality of second features 708₂. Based on the plurality of first features 708₁ and the plurality of second features 708₂, a plurality of first alignment vectors 712₁ may be computed. Based on the plurality of first alignment vectors, a first transformation 714₁ may be determined, which may be used to transform the first laser speckle contrast image 704₁ to register the first laser speckle contrast image with the second laser speckle contrast image 704₂, resulting in a first registered laser speckle contrast image 718₁. As was discussed above, in some embodiments, a transformation may be determined without explicitly detecting features and/or alignment vectors.

Optionally, first weights 716₁ may be determined based on the plurality of first alignment vectors 712₁. A weight may be correlated, preferably inversely correlated, to a length of a representative alignment vector, e.g., a maximum, an average or a median alignment vector, or to e.g. an average or median length of the plurality of first alignment vectors. In an embodiment where the image is divided into a plurality of regions and a transformation is determined for each region, as explained above with reference to FIG. 6D, a weight may be determined for each region based on registration parameters associated with that region or a single weight may be determined for the entire image, e.g. based on a representative parameter, e.g. the largest, average, or median displacement. Determination of a weighted average is discussed in more detail below with reference to FIGS. 9A and 9B.

The first registered laser speckle contrast image 718₁ may then be combined with the second laser speckle contrast image 704₂, resulting in a first combined laser speckle contrast image 720₁. The combined laser speckle contrast image can be, e.g., a pixel-wise average or maximum of the first registered laser speckle contrast image 718₁ and the second laser speckle contrast image 704₂. Optionally, first weights 716₁ and/or second weights 716₂ may be used to determine a weighted average.

At a third time instance t=t₃, step 710₁ may be repeated as step 710₃, resulting in a third raw speckle image 702₃ based on light of the first wavelength, a third laser speckle contrast image 704₃, a third correction image 706₃ based on light of the at least second wavelength, and a plurality of second features 708₃. Based on the plurality of second features 708₂ and the plurality of third features 708₃, a plurality of second alignment vectors 712₂ may be computed. Based on the plurality of second alignment vectors, a second transformation 714₂ may be determined. The second transformation may be used to transform the first combined laser speckle contrast image 720₁ to register the first combined laser speckle contrast image with the third laser speckle contrast image 704₃, resulting in a first registered combined laser speckle contrast image 722₁. The first registered laser speckle contrast image 718₁ may then be combined with the second laser speckle contrast image 704₂, resulting in a first combined laser speckle contrast image 720₁.

The first registered combined laser speckle contrast image 720₁ may then be combined with the third laser speckle contrast image 704₃, resulting in a second combined laser speckle contrast image 720₂. Thus, the second combined laser speckle contrast image may comprise information from the first laser speckle contrast image 704₁, the second laser speckle contrast image 704₂, and the third laser speckle contrast image 704₃. By repeating these steps, the n^{th} image may comprise information from all previous n−1 images. Preferably, the weighing may then be skewed to give recent images a higher weight then older images. This embodiment is particularly useful for streaming video, where each captured frame is processed and outputted with a minimal delay. Another advantage of this method is that between two subsequent frames, motion may be assumed to be relatively small, which may speed up processing, and which may allow a wider range of algorithms to be used, as some algorithms may work less well for large motions. In general, feature-based algorithms may be more reliable for relatively large displacements.

FIG. 7B depicts a flow diagram for an alternative method for laser speckle contrast imaging combining more than two raw speckle images according to an embodiment. In this embodiment, a predetermined number of images is combined into a single combined image. Steps 752₁₋ₙ-760₁₋ₙ relating to image acquisition, computation of speckle contrast images, and determination of features, may be the same as steps 702₁₋ₙ-710₁₋ₙ, explained above with reference to FIG. 7A.

However, different from the method depicted in FIG. 7A, all alignment vectors 762_{1,2} are determined relative to a single reference image, e.g. the first or last image in the sequence of n images. In the depicted example, the images are registered with the n^{th} image. Thus, by applying transformations 764_{1,2} to the first and second speckle contrast images, respectively, the first and second speckle contrast images are registered with the n^{th} speckle contrast image. Consequently, the weights 766_{1,2} are also determined based on a transformation parameter, e.g. average displacement, relative to the n^{th} image. In a final step, all n registered speckle contrast images may be combined into a single combined speckle contrast image 770.

The method depicted in FIG. 7B may result in a combined image having a higher image quality than the method depicted in FIG. 7A, but at the cost of a larger delay between image capture and display. Thus, this method is especially advantageous for recording snap shots.

In an embodiment, the method depicted in FIG. 7B may be applied on a sliding group of images, e.g. the last n images of a video feed. To keep the time delay low, in this case, n should preferably be not too large, e.g. n may be about 5-20 when the frame rate is e.g. 50-60 fps. Of course, the number of frames which may be processed also depends on the hardware and the algorithm, so larger amounts of frames may still be feasible. Thus, some of the advantages of both methods may be combined. An advantage of using a relatively small number of frames is that dynamic phenomena, e.g. the effect of a heartbeat, may be imaged. An advantage of a larger amount of frames, is that such transient effects may be filtered out, especially when the number of frames is selected to cover an integer multiple of heartbeats and/or respiration cycles.

FIG. 8 depicts a flow diagram for computing a corrected laser speckle contrast image according to an embodiment. In general, one may be interested in the relative motion of one or more objects in the target area relative to one or more other objects in the target area; for example, motion of a bodily fluid or red blood cells relative to a tissue. In such a case, noise in a signal derived from the moving object (desired signal) may be compensated by a signal derived from a reference object (reference signal). The underlying principle is that the desired signal may comprise a first component based on the motion of the quantity of interest relative to the reference object, and a second component based on the motion of the entire target area relative to the camera. The reference signal may comprise only, or mainly, a component based on the motion of the entire target area relative to the camera. The reference signal may therefore be correlated to the second component of the desired signal. This correlation may be used to correct or compensate the desired signal. For example, a correction term based on the signal strength of the reference signal may be added to the desired signal.

In the embodiment depicted in FIG. 8, a target area is illuminated with coherent light of a first wavelength 802, e.g. red or infrared light, and illuminated with coherent light of a second wavelength 812, e.g. green or blue light. Preferably, the light of the first wavelength is mostly scattered by the object or fluid of interest, e.g. blood. Preferably, the light of the second wavelength is mostly scattered by the surface of the target area and/or mostly absorbed by the object or fluid of interest. Preferably, the second wavelength is selected such that the reflection of the second wavelength by blood is at least 25%, at least 50%, or at least 75% less than the reflection by tissue. Preferably, the target area is illuminated with light of the first and second wavelengths simultaneously.

The scattered light of the first wavelength may result in a first raw speckle image, which may be captured 804 by a first image sensor. The scattered light of the second raw speckle image may be captured 814 by a second image sensor, preferably different from the first image sensor. The first raw speckle image may be referred to as a desired signal raw speckle image, while the second raw speckle image may be referred to as a reference signal image or a correction signal image.

Based on the first raw speckle image, a first speckle contrast image may be calculated 806. Based on the second raw speckle image, a second speckle contrast image may be calculated 806. Preferably, the speckle contrast is calculated in the same way for the first and second raw speckle images. Speckle contrast may be calculated, for example, in the way that has been explained above with reference to FIG. 1 and step 304 of FIG. 3A.

In a next step 808, a corrected speckle contrast image may be calculated based on the first and second speckle contrast images. Calculating a corrected speckle contrast image may comprise e.g. adding a correction term or multiplying by a correction factor. A correction term or correction term may be based on a determined amount of speckle contrast in the speckle contrast image in comparison with a reference amount of speckle contrast. The reference amount of speckle contrast may e.g. be predetermined, or may be determined dynamically based on e.g. the amount of speckle contrast in a number of preceding second contrast images, or based on an speckle contrast image with very little motion as determined by e.g. the motion correction algorithm as has been described above.

The corrected speckle contrast image may then be stored 810 for further processing, e.g. reregistration or realignment and temporal averaging as was explained with reference to FIGS. 3A and B. The second raw speckle image and/or the second speckle contrast image may also be stored 818 for further processing, e.g. to determine alignment vectors in a plurality of second raw speckle images to reregister or realign a plurality of simultaneously captured corrected speckle contrast images. In an embodiment, steps 802-818 may replace steps 332-336 in FIG. 3B.

Thus, it is an advantage of the methods in this disclosure that the second wavelength image may be used both for multi-spectral coherent correction (or ‘dual laser correction’), as explained with reference to FIG. 8, and for registering speckle contrast images as explained with reference to FIGS. 3A and B.

FIG. 9A schematically depicts determining a motion-compensated speckle contrast image based on a weighted average, according to an embodiment. For each correction image 902_{1-N}, a transformation size may be determined based on registration parameters defining the determined transformation, and/or on the plurality of alignment vectors. For example, the transformation size may be based on the lengths of the plurality of alignment vectors or a statistical representation, e.g. an average 904_{1-N} thereof, or on a matrix norm of a matrix representing the transformation. Non-feature-based image registration methods typically provide only a set of global registration parameters, or a limited set of registration parameters; in such cases, weights may be derived directly from the registration parameters rather than from a set of alignment vectors. Optionally, different types of transformations, e.g., translations, rotations, and scaling, may be given different weights.

The combined speckle contrast image 906 may be a weighted average of the speckle contrast images in the sequence of registered speckle contrast images, each image being weighted with a weight parameter w′.

The weight parameter w′ may be determined based on the alignment vectors

$$‖ {p}_{i}^{′} - {p}_{i} ‖ ,$$

with a high displacement corresponding to a small weight and vice versa, e.g. as defined in equation (11):

$${w}^{′} = \frac{1}{P} {\sum }_{i = 1}^{P} \frac{1}{‖ {p}_{i}^{′} - {p}_{i} ‖} \qquad ( 11 )$$

Here, the alignment vectors may be defined by a total of P points pᵢ′ which are defined by the coordinates (xᵢ′, yᵢ′) on a reference image and points pᵢ which are defined by the coordinates (xᵢ, yᵢ) on the image that is to be transformed to be registered to the reference image.

The weight parameter w′ may also be determined based on a dense or sparse optical flow parameter defining the optical flow between a first image, typically a reference image, and a second image. For example, a weight may be inversely correlated to the average optical flow over all pixels in the image or in a region of interest in the image, e.g. as defined in equation (12):

$${w}^{′} = \frac{1}{W \cdot H} {\sum }_{i , j} \frac{1}{‖ {v}_{ij}^{′} ‖} \qquad ( 12 )$$

Here, vᵢⱼ′ is the optical flow of a pixel (i, j) comprising an x and an y component of the optical flow, and W and H are respectively the width and height in pixels of the reference image or the reference region of interest.

Alternatively, a weight parameter may be determined for each pixel, e.g., based on the optical flow per pixel as defined in equation (13):

$${w}_{ij}^{′} = \frac{1}{‖ {v}_{ij} ‖} \qquad ( 13 )$$

Instead of determining a weight parameter for each pixel or for the image as a while, a weight parameter may also be determined for predefined regions of the image such as a rectangular or triangular mesh. For instance, a weight parameter may be based on the average optical flow in a corresponding predefined region.

The weight parameter may also be determined based on the speckle contrast values sᵢⱼ′, preferably the weight parameter being proportional to the speckle contrast magnitude, e.g. as defined in equation (14):

$${w}^{′} = \frac{1}{W \cdot H} {\sum }_{i , j} ‖ {s}_{ij}^{′} ‖ \qquad ( 14 )$$

Here, sᵢⱼ′ is the speckle contrast of the reference image and W and H are respectively the width and height in pixels of the reference image or the reference region of interest. An advantage of using the speckle contrast is that any noise occurring in a small temporal window on the speckle contrast would blur the speckle contrast. Such noise could be due to motion of e.g. the images object or the camera, or to other sources such as loose fibre connections. An advantage of using weights based on alignment vectors or optical flow is that they may have a higher temporal resolution for LSCI, while a speckle contrast-based weight may lag behind, especially when the perfusion is increasing.

Alternatively, the weight parameter can be determined based on a speckle contrast per pixel.

$${w}_{ij}^{′} = ‖ \frac{1}{{s}_{ij}} ‖ \qquad ( 15 )$$

Alternatively, the weight parameter can be determined based on the average speckle contrast in predefined regions such as a square grid or triangular mesh. Weight parameters may also be determined for dynamically determined regions, where regions may e.g. be determined based on the detected motion.

In an embodiment, various of these weights may be combined. For example, the weights could be normalised and added, multiplied, or compared, selecting e.g. the lowest weight. Alternatively, a first weight, e.g. based on speckle contrast values, may be used to filter out images that do not meet a predefined quality standard, e.g. images having a reduction in contrast magnitude exceeding a predetermined threshold receiving a weight of 0 and all other images receiving a weight of 1. Subsequently, an optical flow or displacement based weight may be used to determine a weighted average of the images that have not been filtered out.

A weighted average may be determined by using a single weight factor per image using a buffer of N images Imgₖ and a buffer of corresponding N weight factors wₖ, with k=1, 2 . . . , N. For every new image that is acquired, the following steps may be performed:
1) adding the new image to the buffer as Img_{N+1}, which may be selected as a reference image;
2) removing a first weight factor w₁ and a first image Img₁ from the buffer;
3) applying a geometrical transformation to the other images Img₂, Img₃, . . . , Img_{N} in the buffer to register them to reference image Img_{N+1}. If the images have been previously registered to e.g. a previous reference image, the same transformation may be applied to all images Img_{2-N}. If unregistered images are stored in the buffer, a transformation may be determined for and applied to each image in the buffer separately;
4) computing a weight factor w_{N+1} as described above and adding it to the buffer;
5) normalizing weight factors w₂ to w_{N+1}, e.g. according to equation (16):

$${w}_{k}^{′} = \frac{{w}_{k}}{{Σ}_{i = 2}^{i = N + 1} ( {w}_{i} + {β}_{1} )} \qquad ( 16 )$$

or according to equation (17):

$${w}_{k}^{′} = \frac{{w}_{k}}{{Σ}_{i = 2}^{i = N + 1} ( {w}_{i} - {w}_{min} + {β}_{2} )} \qquad ( 17 )$$

Here, β₁ is a constant that can be positive or negative, wₘᵢₙ is defined as the minimum weight in the buffer and β₂ is a constant that should be greater than zero. To avoid negative weights and divisions by zero, any weight that is negative or zero can be set to a small positive number. The advantage of using the second normalization with wₘᵢₙ included is to increase the influence of the weight factor. Increasing β₁ or β₂ will decrease the influence of the weight factor and cause the algorithm to behave more like an averaging algorithm while decreasing β₁ or β₂ Will increase the influence of the weight factor. These constants can be predetermined based on the application.
6) computing a high-quality combined image Img_{N+1}′ by computing a weighted average, using the previously computed weights, e.g. as defined in equation (18):

$${Img}_{N + 1}^{′} ( i , j ) = {\sum }_{k = 2}^{N + 1} {w}^{′} \cdot {Img}_{k} ( i , j ) \qquad ( 18 )$$

Here, i and j are used to index the pixels for the images.

In an alternative embodiment, the image buffer may only comprise the combined image. In such an embodiment, a weighted average may be determined by using a buffer of N weight factors wₖ, with k=1, 2 . . . , N corresponding to the N most recent images, and a combined image Img_{N}′ based on the N most recent images. For every new image Img_{N}+1 the following steps may be taken:
1) adding the new image to the buffer as Img_{N+1}, which may be selected as a reference image;
2) removing a first weight factor w₁ from the buffer;
3) applying a geometrical transformation to the stored image Img_{N}′ to register it to the reference image Img_{N}+1.
4) computing a weight factor w_{N+1} as described above and adding it to the buffer;
5) normalizing weight factors w₂ to w_{N+1}, e.g. according to equation (16) or (17);
6) computing a high-quality combined image Img_{N+1}′ by computing a weighted average of the stored image Img′_{N} and the reference image Img_{N+1}, e.g. as defined in equation (19):

$${Img}_{N + 1}^{′} ( i , j ) = ( 1 - {w}_{N + 1} ) \cdot {Img}_{N} ( i , j ) + {w}_{N + 1} \cdot {Img}_{N + 1} ( i , j ) \qquad ( 19 )$$

where i and j are used to index the pixels for the images; and
7) removing Img_{N}′ from the buffer and adding Img_{N+1}′ to the buffer.

The advantage of this algorithm is that it is much faster to compute since only one geometrical transformation has to be applied and the amount of processing steps is lower. The size of the buffer where the weight factors are stored determines how large the influence of the history images are compared to the influence of the new image. When the buffer is small, the new image will be more present in the final image while if the buffer is large the new image will be less present while images with a high weight factor will be more present.

As was explained above, the weights may be determined for an image as a whole, for each pixel in an image, or for predetermined or dynamically determined regions in an image. Similarly, a single transformations may be applied to each image as a whole, each pixel may be individually transformed, or the transformation may be determined for an applied to predetermined or dynamically determined regions. Thus, the weights may be defined as scalars, as matrices, or in different format.

An advantage of an algorithm using geometrical transformations based on e.g. rectangular or triangular meshes, and using weights determined per mesh segment, is that such an algorithm may be more robust to registration errors while still being able to correct locally for noise such as local motion.

A weight based on the amount of displacement or amount of transformation may be determined quickly for each image, independent of other images. Images with a large amount of displacement are generally noisier, and may therefore be assigned a lower weight, thus increasing the quality of the combined image.

Alternatively or additionally, a normalised amount of speckle contrast or an amount of change in speckle contrast relative to one or more previous and/or subsequent images in the sequence of first speckle contrast images may be determined for each first raw speckle image. In that case, the weighted average may be determined using weights based on the determined normalised amount of speckle contrast or the determined change in speckle contrast associated with the respective first speckle contrast image.

Weights based on differences or changes in speckle contrast, especially sudden changes, may be indicative for image quality. Typically, speckle contrast, and hence these weights, may be affected by various factors in the entire system, e.g. motion of the camera relative to the target area, movement of fibres or other factors influencing the optical path length, loose connections, or fluctuating lighting conditions. Hence, using weights based on speckle contrast, a higher quality combined image may be obtained. Typically, speckle contrast is determined in relative units, so weights may be determined by analysing a sequence of raw speckle images. As speckle contrast is inversely correlated with perfusion, speckle contrast-based perfusion units could similarly be used.

Preferably, the images may be normalized in such a way that the relation between the speckle contrast and weight is a linear with a constant. In that case, speckle-contrast-based correction could be more real-time, because each image might be normalized directly and without reference to a temporal window of images. Alternatively, an incremental average might be used.

FIG. 9B schematically depicts determining a motion-compensated speckle contrast image based on a weighted average, according to an embodiment. In this example, a cyclic image buffer 910 is used. When the image buffer is updated, the oldest image is removed 912 from the buffer, the images in the buffer are transformed 914 using the determined image registration parameters, and the new image is added 916 to the buffer. The masks are treated in an analogous way. A cyclic mask buffer 920 of equal size as the image buffer contains the masks associated with the images in the image buffer. When the mask buffer is updated, the oldest mask is removed 922 from the buffer, the masks in the buffer are transformed 924 using the determined image registration parameters, and the new mask associated with the new image is added 916 to the mask buffer. The masks in the mask buffer may be blurred (e.g., using a Gaussian blur or a combination of box filters), to reduce the sensitivity to image registration inaccuracies.

The masks in the updated buffer may then be averaged and normalised 928, resulting in an averaged normalised mask. The average is typically a normal average, but a weighted average can similarly be used, e.g., based on motion parameters as described above with reference to FIG. 9A. That way, the pixels in the mask have a similar or equal weight as the pixels in the weighted average 918 of the speckle contrast images.

In step 918, a weighted average is determined for the images in the image buffer. This weighted average may be referred to as the temporal filter. The weighted average may use several weights. For example, a motion-based weight may be used as described above. This weight may be a local (pixel-based), regional, or global (image-based) weight. Additionally, a mask-based weight may be used. The mask-based weight is typically applied locally, i.e., on a pixel-by-pixel basis.

If an averaged normalised mask has been determined 928, a perfusion value may be computed if the averaged normalised value is lower than a predetermined value (assuming a high mask value indicates a low reliability score), e.g., lower than 0.2. This indicates that in most input images, the pixel value is considered sufficiently reliable. If the averaged normalised value is higher than the predetermined value, the corresponding pixel in the combined image may be given, e.g., an error value, an interpolated value, or no value, as described above.

The pixels for which a perfusion value is computed may then be determined by computing a weighted average, where each pixel in an input image in the image buffer is given a weight based on the associated mask in the mask buffer. In some cases, this is a binary weight, in other cases, a multivalued weight may be used.

This results in a combined image 930, which may then be displayed or used for further processing. In some implementations, the combined image is shown as, e.g., an overlay over a different image (which may be referred to as the underlying image), e.g., the newest image in the image buffer or the associated white-light image. In some cases, the underlying image may be modified based on the mask associated with that image; for example, the intensity of specular reflections in the underlying image may be mitigated by reducing the corresponding pixel values. This may result in a less distracting image.

Similar implementations can be used for other temporal filters, e.g., a decaying buffer. In general, the treatment of the masks in the mask buffer should be analogous to that of the images in the image buffer.

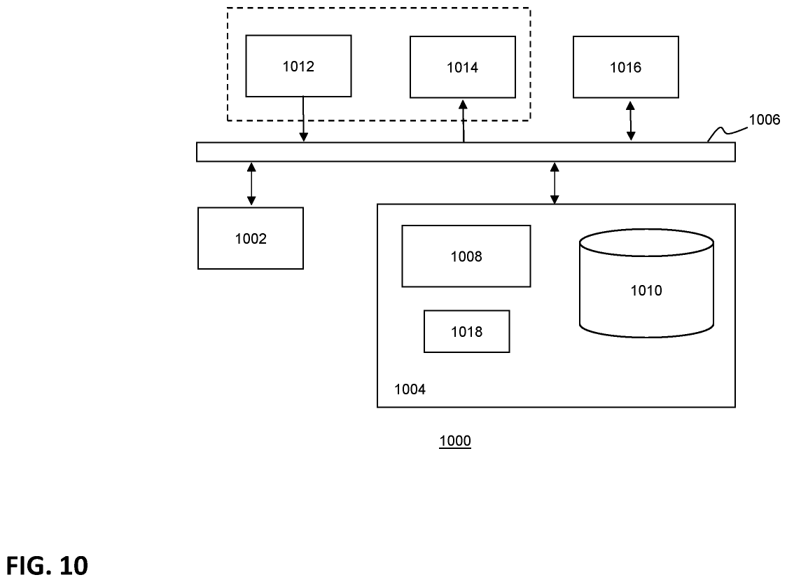

FIG. 10 is a block diagram illustrating exemplary data processing systems described in this disclosure. Data processing system 1000 may include at least one processor 1002 coupled to memory elements 1004 through a system bus 1006. As such, the data processing system may store program code within memory elements 1004. Further, processor 1002 may execute the program code accessed from memory elements 1004 via system bus 1006. In one aspect, data processing system may be implemented as a computer that is suitable for storing and/or executing program code. It should be appreciated, however, that data processing system 1000 may be implemented in the form of any system including a processor and memory that is capable of performing the functions described within this specification.

Memory elements 1004 may include one or more physical memory devices such as, for example, local memory 1008 and one or more bulk storage devices 1010. Local memory may refer to random access memory or other non-persistent memory device(s) generally used during actual execution of the program code. A bulk storage device may be implemented as a hard drive or other persistent data storage device. The processing system 1000 may also include one or more cache memories (not shown) that provide temporary storage of at least some program code in order to reduce the number of times program code must be retrieved from bulk storage device 1010 during execution.

Input/output (I/O) devices depicted as key device 1012 and output device 1014 optionally can be coupled to the data processing system. Examples of key device may include, but are not limited to, for example, a keyboard, a pointing device such as a mouse, or the like. Examples of output device may include, but are not limited to, for example, a monitor or display, speakers, or the like. Key device and/or output device may be coupled to data processing system either directly or through intervening I/O controllers. A network adapter 1016 may also be coupled to data processing system to enable it to become coupled to other systems, computer systems, remote network devices, and/or remote storage devices through intervening private or public networks. The network adapter may comprise a data receiver for receiving data that is transmitted by said systems, devices and/or networks to said data and a data transmitter for transmitting data to said systems, devices and/or networks. Operation modems, cable operation modems, and Ethernet cards are examples of different types of network adapter that may be used with data processing system 1000.

As pictured in FIG. 10, memory elements 1004 may store an application 1018. It should be appreciated that data processing system 1000 may further execute an operating system (not shown) that can facilitate execution of the application. Application, being implemented in the form of executable program code, can be executed by data processing system 1000, e.g., by processor 1002. Responsive to executing application, data processing system may be configured to perform one or more operations to be described herein in further detail.

In one aspect, for example, data processing system 1000 may represent a client data processing system. In that case, application 1018 may represent a client application that, when executed, configures data processing system 1000 to perform the various functions described herein with reference to a “client”. Examples of a client can include, but are not limited to, a personal computer, a portable computer, a mobile phone, or the like.

In another aspect, data processing system may represent a server. For example, data processing system may represent an (HTTP) server in which case application 1018, when executed, may configure data processing system to perform (HTTP) server operations. In another aspect, data processing system may represent a module, unit or function as referred to in this specification.

The terminology used herein is for the purpose of describing particular embodiments only and is not intended to be limiting. As used herein, the singular forms “a,” “an,” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

1. A method of motion-compensated laser speckle contrast imaging comprising:
exposing a target area to coherent first light of a first wavelength, the target area including living tissue;
capturing at least one sequence of images, the at least one sequence of images comprising first speckle images, the first speckle images being captured during the exposing step;
determining one or more registration parameters of an image registration algorithm for registering the first speckle images with each other based on the at least one sequence of images captured in said capturing step, the determining of the one or more registration parameters comprising:
determining transformed images by applying a transformation to the images in the at least one sequence of images captured in said capturing step and determining the one or more registration parameters based on a comparison of the transformed images; and
determining registered spatial speckle contrast images, the determining of the registered spatial speckle contrast images comprising:
determining registered first speckle images by registering the first speckle images based on the one or more registration parameters and the image registration algorithm, and determining the registered spatial speckle contrast images based on the registered first speckle images; or
determining first spatial speckle contrast images based on the first speckle images, and determining registered spatial speckle contrast images by registering the first spatial speckle contrast images based on the one or more registration parameters and the image registration algorithm; and
determining a combined speckle contrast image, the determining of the combined speckle contrast image comprising computing a weighted average of the registered spatial speckle contrast images, wherein a weight of an image, of an image region or of pixels in the image is based on one or more of: the one or more registration parameters, or differences and changes in speckle contrast.

2. The method as claimed in claim 1, wherein the determining of the one or more registration parameters is based on images selected from the first speckle images and/or from images associated with the first speckle images and/or from images derived from the first speckle images or from the images associated with the first speckle images.

3. The method as claimed in claim 2, wherein the registration parameters are based on a similarity measure of pixel values in one or more pixel groups in each of the at least one sequence of images.

4. The method as claimed in claim 1, wherein the transformation comprises one or more of: a Fourier transformation, a Mellin transformation, a Laplace transformation, a Radon transformation, and a log-polar coordinate transformation.

5. The method as claimed in claim 1, wherein the transformation is a transformation to a frequency domain, and wherein the comparison of the transformed images comprises:
determining a cross-correlation of the transformed images;
determining a transformation of the cross-correlation to the spatial domain; and
determining a peak in the cross-correlation in the spatial domain.

6. The method as claimed in claim 1, wherein the transformation is a log-polar coordinate transformation;
the comparison of the transformed images comprises determining a shift on the transformed images relative to each other; and
the determining of the registration parameters comprises determining a rotation and/or a scaling based on the determined shift.

7. A method of motion-compensated laser speckle contrast imaging comprising:
exposing a target area to coherent first light of a first wavelength, the target area including living tissue;
capturing at least one sequence of images, the at least one sequence of images comprising first speckle images, the first speckle images being captured during the exposing step;
determining one or more registration parameters of an image registration algorithm for registering the first speckle images with each other based on the at least one sequence of images captured in said capturing step, the determining of the one or more registration parameters comprising:
selecting, based on pixel coordinates, one or more first pixel groups in a first image of the at least one sequence of images,
selecting, based on pixel coordinates, one or more second pixel groups in a second image of the at least one sequence of images, the one or more first pixel groups having a different size than the one or more second pixel groups, and
determining the registration parameters based on a similarity measure of pixel values in the one or more first pixel groups and the one or more second pixel groups; and
determining registered spatial speckle contrast images, the determining of the registered spatial speckle contrast images comprising:
determining registered first speckle images by registering the first speckle images based on the one or more registration parameters and the image registration algorithm, and determining the registered spatial speckle contrast images based on the registered first speckle images; or
determining first spatial speckle contrast images based on the first speckle images, and determining registered spatial speckle contrast images by registering the first spatial speckle contrast images based on the one or more registration parameters and the image registration algorithm; and
determining a combined speckle contrast image, the determining of the combined speckle contrast image comprising computing a weighted average of the registered spatial speckle contrast images, wherein a weight of an image, of an image region or of pixels in the image is based on one or more of: the one or more registration parameters, or differences and changes in speckle contrast.

8. (canceled)

9. The method as claimed in claim 1, the method further comprising:
determining a plurality of masks for the first speckle images, each of the plurality of masks being associated with a respective first said speckle image, and each of the plurality of masks associating a reliability score with one or more pixels in the associated first speckle image;
determining a plurality of registered masks by registering the plurality of masks, based on the one or more registration parameters and the image registration algorithm; and
determining the combined speckle contrast image based on the plurality of registered masks.

10. (canceled)

11. The method as claimed in claim 9,
wherein the determining of the plurality of masks comprises identifying deviating input pixel values, and/or deviating speckle contrast values, values above a predetermined absolute or relative upper threshold value or values below a predetermined absolute or relative lower threshold value; and/or
the determining of the plurality of masks comprises identifying pixels not representing living tissue based on an image recognition algorithm.

12. (canceled)

13. The method as claimed in claim 9, wherein masked pixels have a weight based on the reliability score associated with the pixel.

14. (canceled)

15. (canceled)

16. The method as claimed in claim 1, the method further comprising:
exposing the target area to second light of one or more second wavelengths, wherein the exposing to the second light is alternated with the exposing to the first light or is simultaneous with the exposing to the first light;
the at least one sequence of images comprises a sequence of second images, the second images being captured during said exposing with the second light; and
the one or more registration parameters are based on the sequence of second images or from images derived from the second images, each of the second images or the images derived from the second images being associated with a first speckle image.

17. (canceled)

18. (canceled)

19. The method as claimed in claim 1, wherein the one or more pixel groups represent predetermined features in the sequence of images.

20. (canceled)

21. The method as claimed in claim 1, wherein a pixel group in a first image from the at least one sequence of images is smaller than a pixel group in a second image from the at least one sequence of images.

22. The method as claimed in claim 1, wherein determining one or more registration parameters comprises:
determining a plurality of associated pixel groups based on the similarity measure, each said associated pixel group belonging to a different image from the at least one sequence of images,
determining a plurality of alignment vectors based on positions of the associated pixel groups relative to respective ones of said images from the at least one sequence of images, the alignment vectors representing motion of the target area relative to an image sensor; and
determining the one or more registration parameters based on the plurality of alignment vectors.

23. The method as claimed in claim 1, further comprising:
dividing each image of the first speckle images, respectively first speckle contrast images, and each image in the at least one sequence of images into a plurality of regions; and wherein
determining the one or more registration parameters comprises determining registration parameters for each region; and
determining a sequence of the registered first speckle images, respectively first speckle contrast images comprises registering each region of the first speckle image, respectively the first speckle contrast image, based on the transformation based on a corresponding region in the second image.

24. The method as claimed in claim 1, wherein the target area comprises a perfused organ, and/or comprises one or more blood vessels and/or lymphatic vessels, the method further comprising:
computing a perfusion intensity based on the combined speckle image.

25. (canceled)

26. A computation module for a laser speckle imaging system, comprising a computer readable storage medium having at least a part of a program embodied as computer readable storage code therewith, and a processor coupled to the computer readable storage medium, wherein responsive to executing the computer readable storage code, the processor is configured to perform executable operations, the executable operations comprising:
receiving at least one sequence of images, the at least one sequence of images comprising first speckle images, the first speckle images having been captured during exposure of a target area to coherent first light of a first wavelength, the target area including living tissue;
determining one or more registration parameters of an image registration algorithm for registering the first speckle images with each other based on the at least one sequence of images,
the determining of the one or more registration parameters comprising:
determining transformed images by applying a transformation to images in the at least one sequence of images and determining the one or more registration parameters based on a comparison of the transformed images; or
the determination determining of the one or more registration parameters comprising:
selecting, based on pixel coordinates, one or more first pixel groups in a first image of the at least one sequence of images,
selecting, based on pixel coordinates, one or more second pixel groups in a second image of the at least one sequence of images, the one or more first pixel groups having a different size than the one or more second pixel groups, and
determining the one or more registration parameters based on a similarity measure of pixel values in the one or more first pixel groups and the one or more second pixel groups; and
determining registered spatial speckle contrast images, the determining of the registered spatial speckle contrast images comprising:
determining registered first speckle images by registering the first speckle images based on the one or more registration parameters and the image registration algorithm, and determining the registered spatial speckle contrast images based on the registered first speckle images; or
determining first spatial speckle contrast images based on the first speckle images, and determining registered spatial speckle contrast images by registering the first spatial speckle contrast images based on the one or more registration parameters and the image registration algorithm; and
determining a combined speckle contrast image, the determining of the combined speckle contrast image comprising computing a weighted average of the registered spatial speckle contrast images, wherein a weight of an image, of an image region or of pixels in the image is based on one or more of: the one or more registration parameters, or differences and changes in speckle contrast.

27. The computation module as claimed in claim 26,
wherein the at least one sequence of images comprises a sequence of second images, the second images being captured during exposure with second light, the second light having one or more second wavelengths the exposure to the second light is alternated with the exposure to the first light or is simultaneous with the exposure to the first light; and
wherein the one or more registration parameters are determined based on the sequence of second images, each of the second images being associated with a one said first speckle image.

28. (canceled)

29. A hardware module for an imaging device, comprising:
a first light source for exposing a target area to coherent first light of a first wavelength, the target area including living tissue;
at least one image sensor system for capturing at least one sequence of images, the at least one sequence of images comprising first speckle images, the first speckle images being captured during exposure with the coherent first light; and
a computation module as claimed in claim 26.

30. The hardware module as claimed in claim 29, further comprising:
a second light source for illuminating, simultaneously or alternatingly with the first light source, the target area with a second light of at least a second wavelength, different from the first wavelength;
wherein the at least one image sensor system is further configured to capture a sequence of second images, the second images being captured during exposure with the second light; and
registration parameters are based on the sequence of second images, each of the second images being associated with a first speckle image.

31. (canceled)

32. (canceled)

33. A medical imaging device comprising a hardware module as claimed in claim 29, the medical imaging device being one of: an endoscope, a laparoscope, a surgical robot, a handheld laser speckle contrast imaging device or an open surgical laser speckle contrast imaging system.

34. A non-transitory computer-readable storage medium having stored thereon a computer program or suite of computer programs comprising at least one software code portion, the at least one software code portion, when run on a computer system, being configured for executing the method steps of:
receiving at least one sequence of images, the at least one sequence of images comprising first speckle images, the first speckle images having been captured during exposure of a target area to coherent first light of a first wavelength, the target area including living tissue;
determining one or more registration parameters of an image registration algorithm for registering the first speckle images with each other based on the captured at least one sequence of images,
the determining of the one or more registration parameters comprising:
determining transformed images by applying a transformation to images in the at least one sequence of images and determining the one or more registration parameters based on a comparison of the transformed images; or
the determining of the one or more registration parameters comprising:
selecting, based on pixel coordinates, one or more first pixel groups in a first image of the at least one sequence of images,
selecting, based on pixel coordinates, one or more second pixel groups in a second image of the at least one sequence of images, the one or more first pixel groups having a different size than the one or more second pixel groups, and
determining the one or more registration parameters based on a similarity measure of pixel values in the one or more first pixel groups and the one or more second pixel groups; and
determining registered spatial speckle contrast images, the determining of the registered spatial speckle contrast images comprising:
determining registered first speckle images by registering the first speckle images based on the one or more registration parameters and the image registration algorithm, and determining the registered spatial speckle contrast images based on the registered first speckle images; or
determining first spatial speckle contrast images based on the first speckle images, and determining registered spatial speckle contrast images by registering the first spatial speckle contrast images based on the one or more registration parameters and the image registration algorithm; and
determining a combined speckle contrast image, the determining of the combined speckle contrast image comprising computing a weighted average of the registered spatial speckle contrast images, wherein a weight of an image, of an image region or of pixels in the image is based on one or more of: the one or more registration parameters, or differences and changes in speckle contrast.

35. (canceled)
